(19) United States
(12) Patent Application Publication
(10) Pub. No.: US 2026/0335214 A1
(43) Pub. Date: Oct. 29, 2026

(54) SOLID FORM FOR CREAMS

(71) Applicant: CASTARALO LIMITED, Hong Kong (CN)

(72) Inventors: Delphine ROBERTI, Spa (BE); Gaëtan MILIS, Spa (BE); Laurie MIAS, Hong Kong (CN)

(73) Assignee: CASTARALO LIMITED, Hong Kong (CN)

(21) Appl. No.: 19/487,849

(22) PCT Filed: May 27, 2024
(86) PCT No.: PCT/IB2024/055140

§ 371 Date: Nov. 25, 2025

(30) Foreign Application Priority Data
May 26, 2023 (EP) 23175845.9

(51) Int. Cl. A61K 8/73; A61K 8/06; A61K 8/19; A61K 8/27; A61K 8/34; A61K 8/36; A61K 8/42; A61K 8/44; A61K 8/49; A61K 8/67; A61K 8/92; A61K 8/9789; A61K 8/99; A61Q 19/00

(52) U.S. Cl. CPC A61K 8/732; A61K 8/06; A61K 8/19; A61K 8/27; A61K 8/34; A61K 8/361; A61K 8/42; A61K 8/44; A61K 8/4946; A61K 8/675; A61K 8/73; A61K 8/735; A61K 8/922; A61K 8/9789; A61K 8/99; A61Q 19/00; A61K 2800/28

(57) ABSTRACT

The invention relates to a composition in solid form comprising cosmetic actives for generation of a viscous cosmetic product in a given volume of water and a method for producing the same. Also provided is a kit comprising two or more compositions in solid form and a vial for reconstituting, holding and optionally dispensing a viscous cosmetic product.

### FIELD

The present invention relates to a new formulation of solid compositions which allows for a reduction in packaging and waste.

### BACKGROUND

The cosmetic industry provides cosmetic products in ready-to-use formats and formulations. In general, cosmetic and/or cleansing products for the skin, body and hair are in the form of aqueous dispersions or emulsions provided in relatively large packaging units. The size of the packaging has several disadvantages in that it requires a high amount of packaging material and takes up space in storage and transport. Large amounts of packaging material and space have a negative effect on the environment. In addition, the vials containing the cosmetics are for single use and, even if to some extent recyclable, generate a lot of waste.

US2020/0345590 describes cleansing and conditioning compositions provided as single-use tablets. US2019/147496 describes personal care cleaning products in tablet form, whereby the tablet is intended for direct use. Similarly, WO2019002374 describes solid cleaning cosmetic compositions which disintegrate when the user applies the tablet to his/her hands. U.S. Pat. No. 5,062,994 also provides single use cleaning tablets which disintegrate upon contact with water.

None of the above compositions envisage tablets for reconstitution to a larger volume product. More importantly, none of these documents provide tablets which, when dissolved in water, generate a viscous cosmetic product.

Accordingly, there is a need for cosmetic products that can be distributed as tablets but can be reconstituted to a more or less creamy product.

### SUMMARY

The application provides compositions in solid form comprising one or more cosmetic actives, for reconstitution, with a given volume of water, into a viscous cosmetic product, such as a cream. More particularly, the solid form has a composition which comprises one or more water-soluble and dispersible cosmetic actives agent and additional ingredients which ensure that it can be formulated into a solid form into a stable viscous cosmetic product. The solid form compositions and methods of the invention allow for the provision of solid forms which can generate, upon dissolution in a given amount of water in a vial, a fluid cosmetic, particularly viscous cosmetic products which can but need not be cleansing products.

In particular embodiments, the composition of the solid form comprises one or more water-soluble and dispersible cosmetic actives one or more aggregation controlling agents and further comprises a texturing agent which comprises one or more cold-dispersible modified starches. The compositions further typically comprise one or more water-soluble preservatives and one or more water-soluble pH-modifying agents. The compositions of the invention are characterized in that they are in solid form for dissolving into a cosmetic product of adjustable viscosity with a given volume of water. In particular embodiments, the compositions of the invention comprise 0-30% of one or more aggregation controlling agents, 40-70% of a texturing agent comprising one or more cold dispersible modified starches, 1-10% water-soluble preservatives, 0.1-10% of one or more water-soluble pH-modifying agents and 0.1-25% of one or more water-soluble and dispersible cosmetic active.

Accordingly, in particular embodiments the amount of said water-soluble preservative, water-soluble pH modifying agent, and said water-soluble and dispersible cosmetic actives in said solid form corresponds to the amount of said ingredients required for obtaining an active viscous cosmetic product when reconstituted in a given volume of water.

In particular embodiments, the application envisages compositions wherein said solid form has a mass of between 2 and 50 grams, and said given amount of water is between 10 and 250 ml, preferably wherein said solid form has a mass of preferably between 5-10 grams and said given volume of water is between 35 and 55 ml. In particular embodiments, the application envisages a solid form of 1 g to be dissolved in 5.5-7.5 g of water such as about 6.02 g of water. In particular embodiments, 1 g is dissolved in 7.23 g of water to form 8.23 g or 7.91 ml of cosmetic. Of course larger solid forms can be dissolved in water corresponding to a similar ratio, such as a solid form of 6.95 dissolved in 50.25 ml of water to form 54.98 ml of cosmetic etc.

In particular embodiments, the texturing agent of the composition is a modified starch such as sodium carboxymethyl starch, acetylated distarch adipate or acetylated distarch phosphate as is further detailed herein.

In particular embodiments the one or more cosmetic actives are natural cosmetic actives such as a low, medium or high molecular weight polysacharide, beta glucan, sodium (retinoyl) hyaluronate, bisabolol, niacinamide, biosaccharide gum-1, haematococcus pluvialis extract, panthenol, aloe vera extract, agave extract, allantoin, inulin, glucomannan, zinc salts, and copper salts as is further detailed herein. In particular embodiments, the one or more cosmetic actives comprise a natural skin cleansing agent such as alkyl proteinates salts, more particularly (di) sodium cocoyl glutamate as is further detailed herein.

In particular embodiments of the solid form compositions of the invention said aggregation controlling agent is a maltodextrin. In particular embodiments, where the active is an alkyl proteinate salt such as (di) sodium cocoyl glutamate, a reduced amount of aggregation controlling agent can be used.

In particular embodiments of the compositions of the invention, the water-soluble preservative is selected from one or more of potassium sorbate, sodium benzoate, benzoic acid, levulinic acid, dehydroacetic acid, sorbic acid, and p-anisic acid as is further detailed herein.

In particular embodiments of the compositions of the invention, the pH modifying agent is selected a weak acid or a natural buffers, such as citric acid as is further detailed herein.

In particular embodiments, the composition of the solid form of the invention further comprise one or more essential oils, such as one or more essential oils selected from cardamom, eucalyptus, tangerine, neroli, geranium etc. as is further detailed herein.

The compositions typically also comprise an emulsifier or dispersing agents (such as a sugar or caprylic acid) as is further detailed herein. The inventors have identified that compositions comprising an aggregating agent, a texturing agent, essential oils and an emulsifier or texturing agent can be combined with one or more active agents to generate solid form compositions which can be dissolved in a given volume of water in a vial to generate a fluid but viscous cosmetic composition.

The compositions of the invention have a solid form (and are also referred to herein as solid form compositions or “solid forms”). In particular embodiments, the solid form is a powder, tablet, sphere, a cylinder or a block as is further detailed herein.

In particular embodiments of the compositions of the invention, the addition of the given volume of water to the solid form in a vial results in a cosmetic product which with a weight that is between 5 and 10 times that of the solid form, as is further detailed herein.

In particular embodiments of the compositions of the invention dissolving the solid form into water in a vial results into a viscous cosmetic products such as a face and/or body cream, more particularly a hydrating cream, a face and/or body scrub, or a solar skin protection cream.

The invention also provides, in a related aspect, kits comprising two or more of the compositions in solid form (or solid forms) of the invention and at least one empty vial suitable for holding and optionally dispensing a viscous cosmetic product resulting from the addition of said given volume of water to said solid form. Optionally, the kit further comprises a vial head suitable for dispensing the viscous cosmetic product.

The invention also provides, in a related aspect, a consumer-friendly method for producing a viscous cosmetic product, said method comprising dissolving a composition in solid form according to the invention as described herein, comprising said cosmetic active in a concentration corresponding to the concentration required for said cosmetic product in a given volume, in a volume of water corresponding to said given volume.

The invention also provides, in a related aspect, methods for producing the solid form cosmetic product according to the invention, which basically comprise the steps of
(a) Grinding the ingredients having particle size larger than 0.5 mm; optionally also grinding ingredients having a particle size larger than 0.2 mm.
(b) Blending the ingredients to a homogenous mass;
(c) Providing the homogenous mass in a mold;
(d) Pressing the mass in the mold under heavy but controlled pressure, i.e. higher than atmospheric pressure; and
(e) Unmolding the solid form.

Optionally, in the methods of production of the invention step (d) comprises pressing the mass with a stamp press so as to obtain a logo or a text on the solid form.

The invention further provides methods for making a solid form composition which can be dissolved in a given volume of water to generate a cosmetic product of a desired viscosity, the method comprising combining varying amounts of
a texturing agent comprising one or more cold-dispersible modified starches; and
one or more aggregation controlling agents and/or dispersing agents
   and further including therein
one or more water-soluble and dispersible cosmetic actives;
one or more water-soluble pH-modifying agents; and
one or more preservatives; and
one or more essential oils and an emulsifier;
   wherein the amount of texturing agent, aggregation controlling agent and/or dispersing agent is adjusted to ensure that said composition is a stable solid form but can be easily dissolved in a given volume of water to provide a cosmetic product of a desired viscosity.

### BRIEF DESCRIPTION OF THE FIGURES

FIG. 1 examples of solid form compositions according to particular embodiments of the invention.

FIG. 2 shows/illustrates an embodiment of a method of making the solid form composition of the invention.

FIG. 3 shows/illustrates a method for reconstituting a viscous cosmetic product according to an embodiment of the invention.

FIG. 4 shows/illustrates the effect on viscosity of increasing concentration of the cold-soluble starches used in the compositions of the invention.

FIG. 5 Illustration of the range in viscosity that can be obtained with increasing concentrations of acetylated distarch phosphate.

FIG. 6 Illustration of the range in viscosity that can be obtained with increasing concentrations of sodium carboxymethyl starch.

### DETAILED DESCRIPTION

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

As used herein, the singular forms “a,” “an” and “the” include both singular and plural referents, unless the context clearly indicates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all integer numbers and, where appropriate, fractions subsumed within that range (e.g. 1 to 10% can include 1%, 2%, 3%, 4%, 5%, 6%, 7%, 8%, 9% when referring to, for example, a number of elements, and can also include 1.5%, 2.75%, 3.80% and 7.65%, when referring to, for example, quantities). The recitation of end points also includes the end point values themselves (e.g. from 1% to 10% includes both 1% and 10%). Any numerical range recited herein is intended to include all sub-ranges subsumed therein. This applies to numerical ranges irrespective of whether they are introduced by the expression “from . . . to . . . ” or the expression “between . . . and . . . ” or another expression.

The term “about” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

Provided herein is a novel means of formulating viscous cosmetic products for skin, hair and/or body in a compact and solid format. The solid product contains one or more cosmetic actives in concentrated form, whereby the solid form is dissolved in a specified amount of fluid prior to use. The formulation in a solid form reduces packaging size and waste.

The concept of the invention is that the solid forms can be used to provide cosmetic products with a thicker texture, and this by the end user, i.e. with a defined amount of water in a vial at room temperature and without the need for industrial agitation. The compositions of the invention thus allow easy reconstitution of the viscous product by dissolving the solid form in a fluid such as water in a vial, by manual movement such as stirring or shaking, without requiring a mixing apparatus. In particular embodiment, the reconstitution into the viscous composition by manual shaking occurs within 1-5 minutes, preferably within 1 minute.

The term “solid form” is used herein interchangeably with “solid form composition” and is also referred to herein as “the composition”. The term cosmetic when referring to a composition is known to refer to compositions which are used to improve skin and/or hair appearance and/or fragrance. In the context of the present invention, a cosmetic composition is also envisaged to include cleansing products for hair and/or skin. Examples of envisaged cosmetic compositions are face masks, face and/or body moisturizers, face serum (which can be general or day or night serum), face peelings but also face and/or body wash, face and/or body soap, deodorant, make-up removing gel, cleanser gel, scrub products, sunscreen, hair wash or other hair care products. Such products are for external use only and typically are non-edible.

The term “external use” refers to the application of a composition to the outer surfaces of the human body, including the skin, hair, nails, and mucous membranes, but excluding ingestion, inhalation, or internal use within the body. Examples of external use products or compositions include moisturizers, serums, lotions, creams, shampoos, conditioners, soaps, deodorants, sunscreens. Compositions and products for external use are typically formulated to be safe and effective when applied topical and are not intended for systemic absorption or internal treatment.

The term “non-edible composition” refers to a composition or product not intended for ingestion or internal consumption. These compositions are typically used in personal care, cosmetic, and pharmaceutical products that are applied to, for example, the skin, hair, nails, or other external body surfaces. Non-edible compositions include, but are not limited to, skin care products (such as moisturizers, lotions, creams, and serums), hair care products (such as shampoos, conditioners, and styling agents), personal hygiene items (such as deodorants and sunscreens), and topical medicinal products (such as ointments and creams). Non-edible composition can contain active ingredients, preservatives, and excipients safe and effective for topical use but not suitable for internal consumption or ingestion.

More particularly, the present invention provides for a composition, preferably a non-edible composition, in solid form (also referred to as solid form comprising a composition) comprising one or more cosmetic actives, one or more preservatives and one or more water-soluble pH-modifying agents, wherein the viscosity of the composition is determined by the balance of one or more texturing agents, one or more aggregation controlling agents and typically also one or more dispersing agents or emulsifiers.

The term “cosmetic active” or “cosmetic active ingredient” as used herein refers to a molecule which can be used in the care for the face and body, or to accentuate or change a person's appearance. Cosmetic agents thus include, but are not limited to molecules having a positive effect on the cells of the skin, body and/or hair such as at least one of anti-wrinkle, anti-aging, anti-free radical, cell-promoting, strengthening the barrier function of the skin, protecting the skin and/or hair from external stimuli, skin irritation alleviation, skin soothing, improving skin tone etc.

The nature of the one or more cosmetic actives is not critical to the invention. However, in the context of the present invention the active is preferably a solid. Moreover, the technical effect is particularly advantageous in the context of cosmetic agents that are typically provided as a viscous composition in a vial. Examples of such cosmetics include face and/or body creams, face and/or body scrubs and viscous sunscreen products. Examples of corresponding cosmetic actives include, but are not limited to
Anti-wrinkle, anti-redness, anti-oxidant, collagen-stimulating agents, such as ethyl ascorbic acid, emollients such as petrolatum, lanolin, mineral oil, cocoglycerides, caprylic/capric triglycerides, ceramides and vitamins such as niacinamide, retinoic acid and its salts and esters, panthenol, ascorbic acid and its salts and esters, tocopherols and their esters
Polysaccharides such as beta-glucan, schyzophyllan, hyaluronic acids and their salts, inulins; particularly plant-derived polysaccharides; these typically are used for one or more of short-term soothing, moisturizing, anti-aging, restructuring and touching agents. Exemplary polysaccharides are polysaccharides of L-fucose, D-galactose and galacturonic acid.
humectants such as glycerin or propanediol
vegetal/plant including (micro) algal extracts as Centella asiatica, Aloe vera, heamatococcus Pluvialis, etc. These extracts are known to the skilled person and while their composition may vary depending on the extraction method, are typically known to contain valuable bioactive compounds such as but not limited to polyphenols, triterpenoid saponins, flavonoids, phenolic acids, triterpenic steroids, amino acids and sugars astaxanthin, carotenoids, proteins, lutein, and fatty acids (FAs).
vegetable oils such as the ones from avocado, macadamia, argan, olive or grapeseed essential oils
exfoliating agents such salicylic acid, alpha hydroxy acid (AHA) or fruit acid;

In addition, an active ingredient may be selected from:
sunscreen agent, which can be organic such as benzophenone-3-butyl, mehtoxydibenzoylmethane, ehtylhexyl methoxycinnamate, or inorganic such as titanium dioxide or zinc oxide;
bacteria, such as Lactobacillus, Bificobacterium, Vitreoscilla optionally combined with one or more prebiotic sugars like xylitol
prebiotics, such as actives which can act as hydrating agents and

It will be clear to the skilled person that the nature of the bioactive is not critical to the present invention, except where the nature of the bioactive will significantly impact the texture of the solid form as detailed herein.

In particular embodiments, the compositions of the present invention further comprise one or more non-soluble cosmetic actives such as a scrubbing agent. Examples of scrubbing agents include but are not limited to powdered grains from seed, kernels, bamboo, coffee, rice.

In particular embodiments, the cosmetic active agent in the compositions described herein comprises at least one or more of a cosmetic active such as but not limited to a low or high molecular weight polysaccharide, panthenol, aloe vera extract, agave extract, allantoin, inulin, zinc salts, copper salts, hyaluron, etc. Preferably, the active is a natural compound. In particular embodiments, the composition is a skin care composition and comprises inulin, a carbohydrate found in plants such as chicory root, as an active ingredient to boost the beneficial bacterial flora on the skin. These actives are well known to the skilled person in the field of cosmetics and are commercially available in a water-soluble powder form. In particular embodiments, the active ingredient is not a cleansing product.

As used herein, the term “natural” or “natural compound” or “natural cosmetic actives” refers to ingredients derived from natural sources, such as plants, minerals, or other organic materials. These natural compounds are typically obtained through extraction or purification processes that preserve their original chemical composition and biological activity and properties. In embodiments, natural compounds are free from synthetic additives. Some examples of natural compounds mentioned above include aloe vera extract, and agave extract. Aloe vera extract is derived from the leaves of the aloe vera plant and is known for its soothing and moisturizing properties. Agave extract, obtained from the agave plant, can have hydrating and antioxidant benefits.

As used herein, the term “low molecular weight polysaccharide” refers to a polysaccharide with an average molecular weight below 10000 Da (10 kDa); examples of low molecular weight polysaccharides include but are not limited to low molecular weight hyaluronic acid, dextran sulfate 10. A high molecular weight polysaccharide typically has a weight of above 100 kDa. Medium molecular weight polysaccharides are typically between 10 kDa and 100 kDa. Examples of high molecular weight polysaccharides include but are not limited to cellulose, starch and glycogen. Typically, in the context of the present invention, the polysaccharide is isolated from plants or another natural source.

In particular embodiments, the composition comprises more than one cosmetic active. More particularly, in particular embodiments, the composition comprises different actives with the same or different activities. For instance, a composition for use as a skin care cosmetic can comprise one or more actives with purifying and repairing function in combination with one or more actives, such as a probiotic and a moisturizer and/or in combination with one or more actives with purifying, anti-acne and/or seboregulator function. The nature of the active agent will be selected based on the intended use of the cosmetic, for instance, the following active agents or combinations of active agents can be envisaged:
for moisturizing serum with fresh texture: inulin, beta glucan.
water bio-active anti-wrinkle: hyaluronic acid, inulin, vitamin C derivative (such as ethyl-ascorbic acid).
gentle repairing serum: inulin, allantoin.
make-up removal or cleansing product: sodium cocoyl glutamate, inulin
scrubbing product: powdered grains (such as coffee, rice or other grains), inulin, dipotassium glycyrrhizinate
face mask product: as inulin or maltose or agave extract optionally combined with sodium cocoyl glutamate; or inulin, aloe vera and zinc
sunscreen product: beta glucan and/or glucomannan or schizophyllan or hyaluronic acid/salt.

The active ingredient is preferably present in a concentration of between 0.1-30%, such as between 1-20%, between 2-8%. In some embodiments the active ingredient is present in the composition in a concentration of between 3-15%. In some embodiments the active ingredient is present in the composition in a concentration of between 0.1-3%. In particular embodiments several active ingredients are combined. In these embodiments the total amount of active ingredient may be between 0.1 and 30%.

In particular embodiments, the compositions in solid form of the invention are envisaged for dissolving into a cleansing product and comprise between 5-10% of a probiotic such as inulin and between 7-20% sodium cocoyl glutamate. In particular embodiments, the compositions are envisaged for dissolving into an exfoliating product and comprise between 15-20% powdered grains and optionally also between 5-10% of dipotassium glycyrrhizinate. In particular embodiments, the compositions are envisaged for dissolving into a face or body mask and comprise between 8-15% agave extract, optionally in combination with between 7-12% sodium cocoyl glutamate. In particular embodiments, the compositions are envisaged for dissolving into a face cream and comprise between 10-15% glucan, more particularly alpha glucan. In particular embodiments, the compositions are envisaged for dissolving into a sunscreen product and comprise between 2-7.5% of a sunscreen agent, such as beta-glucan and between 5-15% of a probiotic such as inulin.

As indicated above, the compositions of the invention are provided in solid form which can be dissolved into a cosmetic product which can have variable viscosity. To this end, the compositions of the present invention further comprise one or more aggregation controlling agents, a texturing agent and optionally one or more dispersing agents. The balance of these three agents allow for strong but dispersible tablets which, when dissolved into a given volume of water, provide a viscous cosmetic product. However, in some embodiments a cosmetic active can also have aggregation controlling properties, and be used as an aggregation controlling agent. In particular embodiments, the one or more aggregation controlling agents (such as maltodextrin as detailed below) are present in a concentration of between 10-60%, preferably between 30-50% by weight, the texturing agent (such as the sodium carboxymethyl starch) is present in a concentration of between 15-45%, preferably between 35-45% by weight and the one or more emulsifiers (such as caprylic acid as detailed below), is present in a concentration of 5-10% by weight. Preferably the one or more aggregation controlling agents (such as maltodextrin as detailed below), the texturing agent (such as the sodium carboxymethyl starch) and the one or more emulsifiers (such as caprylic acid as detailed below) make up at least 70%, preferably at least 75% of the total weight of the solid composition. Typically, where the bioactive comprises sodium cocoyl glutamate at a concentration of 10% or more, the concentration of aggregating agent such as maltodextrin is below 25%; where the bioactive does not comprise, or comprises less than 10% sodium cocoyl glutamate, the concentration of aggregating agent is above 25%.

The texturing agent envisaged in the context of the compositions of the present invention comprise one or more cold-dispersible modified starches. The term “cold-dispersible modified starch” refers to a modified starch which is soluble in water at room temperature. Typically, cold-dispersible starches are pre-gelatinized starches. Examples of cold-dispersible modified starches, such as sodium carboxymethyl starch, acetylated distarch adipate or acetylated distarch phosphate, hydroxypropyl distarch phosphate. The latter can be combined with cellulose derivatives such as carboxymethyl cellulose and its salts, hydroxypropylmethyl cellulose, . . . etc. Cold-dispersible modified starches are commonly used in the food industry. The present inventors have however found that cold-dispersible modified starches are particularly suitable for generating solid forms that can be dissolved into viscous compositions.

The term “acetylated distarch adipate” refers to a modified starch compound derived from the chemical modification of native starch, typically obtained from corn or potato starch. The modification process involves acetylation, where acetyl groups (—COCH3) are introduced onto the starch molecules, typically at the C2 and C3 positions of the glucose units. Additionally, adipate groups derived from adipic acid are incorporated into the starch structure. This dual modification enhances the functional properties of the starch, imparting improved stability against heat, acidity, and shear. While the actual structure of acetylated distarch adipate varies, it is as such a well-established molecule known as E1422 and under CAS NO: 63798-35-6.

Acetylated distarch phosphate is known to the skilled person as a modified starch compound derived from the chemical modification of native starch, commonly sourced from corn or potato starch. This modification process involves acetylation, where acetyl groups (—COCH3) are introduced onto the starch molecules, typically at the C2 and C3 positions of the glucose units, alongside the incorporation of phosphate groups derived from phosphoric acid. This dual modification enhances the functional properties of the starch, including improved stability against heat, acidity, and shear. It is also known as E1414 and Cas No. 68130-14-3

The amount of texturing agent provided in the compositions of the invention will depend on the envisaged viscosity of the resulting product. In particular embodiments, the amount of texturing agent in the compositions of the invention is at least 20 weight %, more particularly between 20-80 weight %. In particular embodiments, the amount of texturing agent in the composition is envisaged to be between 30-75 weight %, in some embodiments between 30-70 weight % or between 40-75% by weight. In particular embodiments, for very viscous compositions, the composition comprises between 50-70%, such as between 55 and 60% by weight of a texturing agent. In further embodiments, particularly where a less viscous composition such as a lotion or serum is envisaged the composition comprises between 30-50%, such as between 35-50%, and between 35% and 45% by weight of a texturing agent.

The compositions of the invention may further comprise one or more aggregation-controlling agents. The aggregation-controlling agent help ensure that particles can agglomerate together. Depending on the other components in the composition, a higher or lower amount of the aggregation-controlling agent will be optimal to ensure a composition that is stable in its solid form but can be dispersed in water. The nature of the aggregation-controlling agent is not critical to the invention. Examples of suitable aggregation controlling agents include but are not limited to polyols, such as maltodextrins, sorbitol and xylitol. In particular embodiments a cosmetic active can be used which also has the properties of an aggregation controlling agent, such as is the case for alkyl proteinate salts such as an alkyl glutamate, which are used as cosmetic actives with cleansing properties. In these embodiments, the amount of additional aggregation controlling agent can be reduced. In fact, the inventors have found that the combination of a texturing agent, aggregation controlling agent such as maltodextrin and an alkyl glutamate, such as sodium cocoyl glutamate is particularly suitable for the generation of solid forms which disintegrate well in a predefined amount of water by manual shaking. The amount of texturing agent can be varied between 30-75% to influence the viscosity of the final cosmetic product.

The term “alkyl” is known to the skilled person to refer to a functional group in organic chemistry composed of carbon and hydrogen atoms bonded together in a chain or branched structure. Alkyl groups are derived from alkanes by removing one hydrogen atom, resulting in a free valence. They are characterized by their general formula CₙH₂ₙ₊₁, where ‘n’ represents the number of carbon atoms in the chain. In in embodiments, in alkyl glutamates, an alkyl group attached to a glutamate moiety.

The term “alkyl glutamate” refers to a compound comprising a glutamate molecule with an alkyl group. Glutamate is a naturally occurring amino acid and a key component in proteins. In alkyl glutamates, the alkyl group is typically a hydrocarbon chain derived from alkanes.

In embodiments, said cosmetic active comprises a skin cleaning agent including, but not limited to, sodium cocoyl glutamate which also acts as an aggregation controlling agent such that less aggregation controlling agent is needed compared to a cosmetic active comprising a skin cleaning agent which does not act as aggregation controlling agent or which is different from sodium cocoyl glutamate, as will be described herein below.

Further, in particular embodiments of the compositions of the invention, the aggregation controlling agent is maltodextrin. Depending on the nature of the one or more aggregation controlling agent(s), the amount required to ensure a solid form which will remain solid until use, can vary. For instance, the solid form can comprise at least 7.5% alkyl proteinate salt, such as sodium alkyl glutamate or at least 50% maltodextrin as the only aggregation controlling agent. Alternatively, the solid form can comprise 12.5% or more, and preferably at least 15% of a combination of alkyl proteinate salt and maltodextrin. In further alternative embodiments, the solid form can comprise a combination of 40% native starch and 31% maltodextrin.

The amount of aggregation controlling agents in the composition is envisaged to be between 0-60 weight %. In particular embodiments, the composition comprises between 0-40% preferably between 5-40% of an aggregation controlling agent.

In particular embodiments, the composition of the invention further comprises a dispersing agent and/or disintegrating agent or emulsifier. A dispersing agent is a substance, that is added to a to improve the separation of the particles and to prevent their settling or clumping. Examples of dispersing agents include but are not limited to compositions comprising oligosaccharides such as disaccharides such as sucrose or native starches or boron nitride. In particular embodiments, the dispersing agent can be cane sugar. In particular embodiments, no dispersing agent is required as the texturing agent or some cosmetic actives can also act as dispersant. This can be the case for the cold-soluble modified starches which also have disintegrating effect or corn starch which can act both as a cosmetic to soften the skin and as a disintegrating agent. Typically, the ratio of agglomeration controlling agent vs dispersant should be kept between 1.2 and 1.7, preferably at about 1.56.

The amount of dispersing agent, where required, is envisaged to be between 5-25 weight %. In particular embodiments, the composition comprises between 5-15%, such as about 8% by weight of a dispersing agent.

In particular embodiments, the compositions additionally comprises an emulsifier or dispersant for the oily ingredients in the composition such as the essential oils. An emulsifier or dispersant acts like detergent, dissolving the oil into the water. In these embodiments, the amount of emulsifier added may be between 2-20 weight % of the total composition, such as around 15%. Preferred emulsifiers are organic emulsifiers such as sucro-esters as cetearyl glucoside, sucrose stearate, . . . etc, glycero-esters such as polyglyceryl-10 laurate, polyglyceryl-3 caprylate, . . . etc, and other emulsifiers such as but not limited to cetearyl alcohol, sorbitan olivate and cetearyl olivate. In particular embodiments, the emulsifier or dispersing agent is caprylic ester or polyglyceryl-3-caprylate. Interestingly, some dispersing agents/emulsifiers will also act as antimicrobial agent.

The inventors have found that the combination of a texturing agent; with an active agent and in some embodiments in combination with an aggregation controlling agent and/or a dispersing agent ensures a good equilibrium between the solidity of the solid form composition and its capacity to generate a viscous product containing the relevant concentration of the active when dissolved in a given amount of water by manual shaking.

For instance, in particular embodiments, the compositions comprise about 56% of a texturing agent, and between 5-11% of an aggregation controlling agent. More particularly, a high amount of modified cold-soluble starch will not require the addition of a dispersing agent. In other embodiments, the compositions comprise about 56% of a texturing agent and between 9-20% of an aggregation controlling agent. In further embodiments, for more viscous cosmetic products, the composition comprises around 70% of a texturing agent and about 3% of an aggregation controlling agent.

The compositions of the present invention further comprise a water-soluble pH-modifying agent. The nature of the water-soluble pH modifying agent is not critical to the invention, it can be either a base or an acid. In particular embodiments, the acid is citric acid or lactic acid. In particular embodiments, the base is potassium hydroxide, sodium hydroxide or triethanolamine.

In particular embodiments, the pH modifying agent is an acid, such as citric acid. In order to ensure the skin's barrier function, an acidic environment on the skin is recommended, particularly where a skin-care product for use on older skin is envisaged. Each decade skin becomes more alkaline, resulting in a variety of undesirable changes and skin disorders ranging from wrinkles to pigmentation and even acne. Keeping the pH level of the skin around 5.5, thus slightly acidic, is optimum. Accordingly, in particular embodiments the cosmetic, when reconstituted has a pH of between 4.2-5.5 5, such as 5+/−0.5. In particular embodiments, this is achieved by the combination of active ingredients. In particular embodiments, this is achieved by the inclusion of pH modifying agents such as citric acid.

In particular embodiments the compositions described herein comprise at least one water-soluble pH modifying agent in an amount between 0.1 and 10% in relation to the total weight of the composition, preferably between 0.1 and 5%, more preferably 0.2, 0.3, 0.4, 0.5, 0.6, 0.7, 0.8, 0.9, or between 1 to 10%. The pH modifying agent in the compositions described herein is selected from weak acids or natural buffers. A weak acid is known to the skilled person as an acid chemical which does not dissociate (split into ions) completely in water solution. This means it does not give all its hydrogen ions into the water. Weak acids typically have a pH between 3 and 6. Examples of weak acids include citric acid, acetic acid, lactic acid, ascorbic acid and phosphoric acid. For instance, citric, ascorbic lactic, are preferred to be used at low strength, such as between 0.1 and 10%, such as about 1%, but in other compositions may for instance be up to 12%, or as high as 20%. In particular embodiments the compositions as described herein do not comprise a pH modifying agent.

The composition preferably also contains one or more preservatives or anti-microbial agents. The nature of the preservative is not critical to the invention, other than that it is water-soluble. In particular embodiments, the one or more preservative comprises one or more of benzoic acid and salts of benzoic acid (such as sodium benzoate), proprionic acid and its salts, salicylic acid and its salts, hexa-2, 4 dienoic acid and its salts, potassium sorbate, formaldehyde, paraformaldehyde (or any other preservative listed in the 1223/2009/EC directive) as well as non-listed preservatives such as anisic acid, levulnic acid, glycerol caprylate. In particular embodiments the composition comprises the preservatives is potassium sorbate and sodium benzoate.

In particular embodiments, the compositions described herein comprise at least one water-soluble preservative in an amount between 0.1 and 10% in relation to the total weight of the solid form composition, preferably between 0.1 and 10%, such as between 1-5%, more preferably 1, 2, 3, 4, 5, 6, 7, 8, 9 or 10%. More particularly, the total amount of the one or more preservatives is provided such that the concentration in the final cosmetic (after dissolving the solid form in a given volume of water) is between 0.2 and 1.0%, such as about 0.6%. Typically, for instance an amount of potassium sorbate and of sodium benzoate is added such that the concentration of each preservative in the final cosmetic is 0.3%. In particular embodiments, the water-soluble preservative in the compositions described herein is selected from one or more of potassium sorbate, sodium benzoate, benzoic acid, levulinic acid, dehydro-acetic acid, sorbic acid, p-anisic acid. In particular embodiments, the composition comprises two or more different preservatives, wherein the combined amount of preservatives corresponds to the amount of the preservatives detailed above.

The compositions of the present invention typically comprise one or more fragrances. Different examples of suitable fragrances are known in the art and include essential oils, such as but not limited to essential oils made from natural products such as from citronella, lime, lemongrass, bergamot, cardamom, tangerine, litsea cubeba or May chang, eucalyptus, mint, palmorsa, ventiver, patchouli, jasmine, marigold, lilly of the valley, sugandhmantri, Egyptian or Morrocan néroli etc. . . . . In particular embodiments, essential oils are concentrated, volatile aromatic compounds extracted from plants. In particular embodiments, the composition comprises between 0.01-5% essential oils, preferably between 0.1-5% essential oils, such as 0.3%. In particular embodiments, the composition comprises 0.3% Morrocan néroli essential oil. The compositions may also comprise combinations of essential oils such as eucalyptus and cardamom, mint and bergamot, tangerine and may chang, and tangerine and Egyptian neroli oil.

In particular embodiments, the compositions of the invention comprise 0.8% eucalyptus and 0.16% cardamom. In particular embodiments, the composition comprises 0.8% mint and 0.6% bergamot. In particular embodiments, the composition comprises 1.2% tangerine and 0.08% may chang. In particular embodiments, the composition comprises 1.4% tangerine and 1% Egyptian neroli oil.

As detailed above, the addition of essential oils will typically imply the addition of an emulsifier or dispersant in the concentrations as indicated above. Accordingly with the above concentrations of between 0.5-2.5% essential oils, an amount of between 7-10%, such as about 8% emulsifier is used. More particularly, the amount of emulsifier added to the composition is such that the final amount in the cosmetic is between 0.5 and 2%, such as 1%. P

In particular embodiments the ingredients listed above make up 100% of the composition. However, in particular embodiments, one or more additional ingredients in addition to the ingredients listed above, such as one or more of a coloring agent, an antibacterial agent, an antifungal agent, a vitamin etc.

In particular embodiments, the compositions of the present invention further contain a color pigment or a glittering agent. In particular embodiments, the composition comprises between 0.01-5% of a color pigment.

In particular embodiments most or all of the ingredients of the composition are natural ingredients.

The invention further provides methods for producing compositions in solid form for dissolving in a given volume of water so as to obtain a cosmetic product of a desired viscosity, the method comprising combining
a texturing agent comprising one or more cold-dispersible modified starches
one or more water-soluble pH-modifying agents; and
one or more water-soluble and dispersible cosmetic actives; and, optionally
one or more aggregation controlling agents and one or more dispersing agents
   wherein the amount of texturing agent, aggregation controlling agent and dispersing agent is adjusted to ensure that said composition, can easily be dissolved in a given volume of water provides a cosmetic product of a desired viscosity.

The invention further provides methods for producing compositions in solid form for dissolving in a given volume of water so as to obtain a cosmetic product, which method comprises combining 0.1-25% of one or more water-soluble and dispersible cosmetic actives, one or more preservatives and one or more essential oils with an amount of texturing agent comprising one or more cold-dispersible modified starches varying between 20 and 80%, wherein the amount of texturing agent added to said composition determines the viscosity of the resulting cosmetic product. The method further encompasses optionally adding one or more aggregation-controlling agents and/or, dispersing agents to control the stability of the powder or tablet when dry and the solubility of the powder or tablet in water.

The nature of the texturing agent will of course influence the amount required to obtain the right viscosity. In particular embodiments, the texturing agent is acetylated distarch phosphate and the method comprises including between 3-7% of texturing agent to obtain a cosmetic product with a viscosity of 1000 mPas to 20000 mPas, more particularly about 3% for a viscosity of about 1000 mPas, about 5% for a viscosity of about 6000 mPas and 7% for a viscosity of about 20000 mPas.

In particular embodiments, the texturing agent is sodium carboxymethyl starch and the method comprises including between 6.5-9.5% of texturing agent to obtain a cosmetic product with a viscosity of 1000 mPas to 20000 mPas, more particularly about 6.5% for a viscosity of about 1000 mPas, about 8% for a viscosity of about 6000 mPas and 9.5% for a viscosity of about 20000 mPas.

In embodiments, said composition is in solid form for dissolving in a given volume of water so as to obtain a viscous cosmetic product, wherein the viscosity of the viscous cosmetic product is between about 1000 mPas to 20000 mPas, about 2000 mPas to 19000 mPas, about 3000 mPas to 17000 mPas, about 4000 mPas to 160000 mPas, about 5000 mPas to 15000 mPas, about 6000 mPas to 14000 mPas, about 7000 mPas to 13000 mPas, about 8000 mPas to 12000 mPas.

In particular embodiments, the invention thus provides methods of producing compositions in solid form for dissolving in a given volume of water so as to obtain a viscous cosmetic product which comprises, including in said composition, at least 50% of a texturing agent comprising one or more cold-dispersible modified starches.

The viscosity of a composition can be measured by different methods well established in the art. Viscosity can for instance be measured using a Marsh funnel, a conical-shaped funnel, fitted with a small-bore tube on the bottom end through which mud flows under a gravity head. Microfluidic rheometry determines the dynamic viscosity of fluids in small sample volumes by forcing a liquid sample through a microfluidic channel in a laminar flow. Viscosity can also be measured by applying oscillating vibrations to the sample and monitoring the damping effects of the fluid which can be assessed by monitoring power input, the decay time of oscillations, or changes in the resonated frequency. In particular embodiments, the viscosity is measured by rotational viscometry. Rotational viscometry is the resistance that a fluid shows when being deformed. Rotational viscometers measure the viscosity of the sample by turning a spindle in a cup.

Viscosity can be determined by measuring the torque on a vertical shaft that rotates a spindle. Different types of rotational viscometers are known in the art, depending on whether they can measure low (“L-type”), medium (“R-type”) or high viscosity (“H-type”) samples or two or all of these. In particular embodiments, the viscometer is a Brookfield Rotational viscometer, such as an RV or LV viscometer. Viscosity and the SI physical unit of dynamic viscosity can be expressed (milli) pascal seconds ((m)Pa·s) which is equivalent to N·s/m² or Kg/(m·s) or centipoise (cp). Indeed, the viscosity measures the force necessary to make a surface of fluid (the cosmetic) slide over another immobile surface. The force [N] necessary to move a surface [m²] at a certain speed [m/s] is measured, which is related to a certain thickness of cosmetic [m]; that is to say N/m² per m/s.

In practice, a needle is rotated radially in the cosmetic—the surface of the cosmetic in contact with the needle is mobile, compared to the rest which is immobile. The device measures the force necessary to maintain a constant rotation speed, and translates it into mPa·s. The measurement itself uses the torsion of a spiral spring, which is limited in its movements: it cannot twist less than at rest, which implies that there is a minimum viscosity below which the device measures nothing and on the other hand, it cannot twist until it deforms, which implies that there is a maximum viscosity above which the device must stop measuring. In practice, various needles with small [high viscosity] or large [low viscosity] diameters are used, so that they twist the spring less [small] or more [large] for the same viscosity. The faster the faster the substance moves, the more fluid it is.

The examination of the viscosity curve [y-axis, mPas] vs. speed of rotation of the needle [x-axis, rpm] allows to characterize the viscosity of the cosmetic. This rheofluidity means that the same rotation speed with a small [lower linear speed] or a large [higher linear speed] needle will not give the same viscosity measurement. In a rheofluidizing cosmetic, a lower linear speed [small needle] corresponds to a higher viscosity, and a higher linear speed [large needle] corresponds to a lower viscosity. The needles are referenced by numbers. For instance, in particular embodiments, the needles are referenced by sp4-sp3-sp2-sp1 in order of increasing diameter, and corresponding to lower viscosity ranges: sp4: needle with central shaft of 3.1 mm diam×29.7 mm ht; sp3: needle with central rod of 3.1 mm diam×50.9 mm ht+disc 12.5 mm diam×1.6 mm ht; sp2: needle with central rod 3.1 mm diam×50.9 mm ht+disc 18.7 mm diam×6.7 mm ht.

The slope of this line characterizes the decrease of the curve [in practice, d is negative; and the higher its absolute value, the more fluid the cream becomes when spreading compared to its state at rest]. The origin no of this line reflects in practice the viscosity at rest [mathematically at 1 rpm]. The calculation of the R², typically >0.99, validates the consistency of the measurements with respect to this.

In particular embodiments, the viscosity is measured by a rotational viscometer, by example IKA ROTAVISC SBS, using spindles sp2+sp3+sp4 as described above at temperatures of 20±2 °C.

In particular embodiments, where a creamy product is envisaged the viscosity of the cosmetic product is from about 300 to 500 cps, such as between 360 to 450 cps. In particular embodiments, where a lotion is envisaged, the viscosity is between 120 and 200 cps, such as between 145 to 190 cps, as calculated from the viscosity mPas vs rpm curves and extrapolated to 0.1 rpm.

In particular embodiments, the methods of the invention provide for the generation of a composition in solid form which, when dissolved in a given amount of water results in a cosmetic product which has the viscosity of a gel or cream.

In particular embodiments, the invention provides a composition in solid form comprising
0.1-25% of one or more water-soluble and dispersible cosmetic actives;
30-70% of a texturing agent, such as 40-70% of a texturing agent comprising one or more cold-dispersible modified starches;
and optionally
0-50% of one or more aggregation controlling agents and dispersing agents, such as 0-30% of one or more aggregation controlling agents.

In particular embodiments, the invention provides a composition in solid form comprising
30-50% of a texturing agent comprising one or more cold-dispersible modified starches;
3-8% water-soluble preservatives;
2.5-3.5% of one or more water-soluble pH-modifying agents;
4.5-15% of one or more water-soluble and dispersible cosmetic actives;
0.1-1.0% essential oils;
5-8% of emulsifier; and
30-50% of one or more aggregation controlling agents.

In particular embodiments, the invention provides a composition in solid form, which when dissolved for the generation of a face moisturizer or serum comprising
35-45% of a texturing agent comprising one or more cold-dispersible modified starches;
3-8% water-soluble preservatives;
2.5-3.5% of one or more water-soluble pH-modifying agents;
4.5-15% of one or more water-soluble and dispersible cosmetic actives;
0.1-1.0% essential oils;
5-8% of emulsifier; and
30-45% of one or more aggregation controlling agents, preferably maltodextrin.

In particular embodiments, the invention provides a composition in solid form comprising
35-45% of a texturing agent comprising one or more cold-dispersible modified starches, preferably sodium carboxymethyl potato starch;
3-8% water-soluble preservatives;
2.5-3.5% of one or more water-soluble pH-modifying agents;
4.5-15% of one or more water-soluble and dispersible cosmetic actives;
0.1-1.0% essential oils;
5-8% of emulsifier; and
35-45% of one or more aggregation controlling agents, preferably maltodextrin.

The composition is further characterized in that said composition can be dissolved, preferably by manual agitation, in a given volume of water so as to obtain a cosmetic product of desired viscosity. In preferred embodiments, the resulting composition is a gel or a product of higher viscosity than a fluid, such as a gel or cream. The presence and amount of the aggregation controlling agents and dispersant will be influenced by the nature of the actives. In particular embodiments, where the active has aggregation controlling activity, the amount of (non-cosmetically active) aggregation controlling agent can be lower.

Preferably, said solid forms as described herein above can be dissolved in water to generate a face moisturizer or serum. In particular embodiments, a solid form of about 8-10 g of said composition can be dissolved to a final volume of 50 ml to obtain a cosmetic, such as but not limited to a face moisturizer or serum.

The compositions of the inventions further comprise 1-10% water-soluble preservatives and 0.1-10% of one or more water-soluble pH-modifying agents, as well as essential oils and emulsifiers as detailed herein.

Exemplary combinations of ingredients in the compositions of the present invention, such as those described hereinabove are a cold-soluble modified starch (texturing agent) maltodextrin (aggregation controlling agent), essential oils, emulsifier, preservatives, pH modifiers and cosmetic actives.

In particular embodiments, the composition according to the invention is a solid form which, upon dissolution in water forms a make-up removal gel product, and comprises between 50-60% of a texturing agent comprising starch, such as sodium carboxymethyl potato starch; 4-8% water-soluble preservatives, such as potassium sorbate and sodium benzoate; 0.5-4.5% of one or more water-soluble pH-modifying agents, such as citric acid; and 8-10% of a skin cleaning agent, such as sodium cocoyl glutamate and 5-10% of a cosmetic agent such as inulin; 8-12% of an aggregation controlling agent, such as maltodextrin; 1-2% of essential oils such as tangerine and may chang oil and 7-10% of an emulsifier such as caprylic ester.

In particular embodiments, as detailed above, the amount of aggregation controlling agent can be decreased if the powder or tablet also comprises an active such as sodium cocoyl glutamate, which also controls aggregation.

Accordingly, in particular embodiments, the composition according to the invention is a solid form which, upon dissolution in water forms a cleanser gel product, said solid composition comprising 50-60% of a texturing agent comprising starch, such as sodium carboxymethyl potato starch; 4-8% water-soluble preservatives, such as potassium sorbate and sodium benzoate; 0.5-1.5% of one or more water-soluble pH-modifying agents, such as citric acid; 12-17% of a skin cleaning agent, such as sodium cocoyl glutamate and 5-10% of a cosmetic agent such as inulin; 4-6% of an aggregation controlling agent, such as maltodextrin; 1-2% of essential oils such as mint and bergamot oil and and 7-10% of an emulsifier such as caprylic ester.

In particular embodiments, the composition according to the invention is a solid form which, upon dissolution in water forms a creamy scrubbing product, said solid composition comprising 50-60% of a texturing agent comprising starch, such as sodium carboxymethyl potato starch; 4-8% water-soluble preservatives, such as potassium sorbate and sodium benzoate; 0.5-1.5% of one or more water-soluble pH-modifying agents, such as citric acid; 10-20% of a skin cleaning or scrubbing agent, such as powdered grains and 5-10% of a cosmetic agent such as inulin and optionally up to 10% dipotassium glycyrrhizinate; 0-20% of an aggregation controlling agent (depending on the presence of the dipotassium glycyrrhizinate), such as maltodextrin; 1-2% of essential oils such as eucalyptus and cardamom oil and 7-10% of an emulsifier such as caprylic ester.

In particular embodiments, the composition according to the invention is a solid form which, upon dissolution in water forms a face mask gel product (appearing as a cream), said solid composition comprising 50-75% of a texturing agent comprising starch, such as sodium carboxymethyl potato starch; 4-8% water-soluble preservatives, such as potassium sorbate and sodium benzoate; 0.5-1.5% of one or more water-soluble pH-modifying agents, such as citric acid; 10-20% of a cosmetic agent, such as inulin or maltose or agave extract; and optionally between 5-15% of a cleansing agent such as sodium cocoyl glutamate; 2-20% of an aggregation controlling agent, such as maltodextrin; wherein the composition; 1-2% of essential oils such as tangerine and may chang oil and 7-10% of an emulsifier such as caprylic ester. The application envisages different embodiments of the mask gel, wherein the amount of aggregation controlling agent is reduced as the amount of texturing agent is increased.

In particular embodiments, the composition according to the invention is a solid form which, upon dissolution in water forms a serum product, said solid composition comprising 30-50% of a texturing agent comprising starch, such as sodium carboxymethyl potato starch; 3-8% water-soluble preservatives, such as potassium sorbate and sodium benzoate; 2.0-3.5% of one or more water-soluble pH-modifying agents, such as citric acid; 4.5-10% of a cosmetic agent, such as inulin and/or hyaluronic acid and/or sodium retinoyl hyaluronate; and optionally between 30-50% of an aggregation controlling agent, such as maltodextrin; wherein the composition; 0.15-0.35% of essential oils such as neroli maroc oil and 5-8% of an emulsifier such as caprylic ester. The application envisages different embodiments of the serum, wherein the amount of aggregation controlling agent is reduced as the amount of texturing agent is increased.

In particular embodiments, the composition according to the invention is a solid form, which, upon dissolution in water forms a serum product, said solid composition comprising between 35-50% or 35-45% or 40-45%, preferably 41% of a texturing agent comprising starch, such as sodium carboxymethyl potato starch; 3-8% or 3-6% water-soluble preservatives, such as 1.5-4% potassium sorbate and 1.5 to 3% sodium benzoate, preferably about 2% of each; 2.0-3.5%, preferably 2.8% of one or more water-soluble pH-modifying agents, such as citric acid; 5.5-12% or 8-12% of a cosmetic agent, such as one or more of inulin, hyaluronic acid, preferably about 7% of inulin, about 1.4% of hyaluronic acid; and between 30-45% or 30-40%, preferably about 37% of an aggregation controlling agent, such as maltodextrin; wherein the composition also includes 0.15-0.35%, preferably about 0.3 of essential oils such as neroli maroc oil and 5-8%, preferably 6.6% of an emulsifier such as caprylic ester. Preferred embodiments comprise combinations of the preferred concentrations detailed herein. Preferably, said solid form as detailed above can be dissolved in water to generate a face serum. In particular embodiments, a solid form of about 8-10 g of said composition can be dissolved to a final volume of 50 ml to obtain a cosmetic such as a face serum. In particular embodiments, said face serum will comprise 3-8%, preferably about 5.4% of an aggregation controlling agent, such as maltodextrin between; 4-8%, preferably about 6% of a texturing agent comprising starch, such as sodium carboxymethyl potato starch; 0.2-0.5%, preferably 0.3% water-soluble preservatives, such as 0.15% potassium sorbate and 0.15% sodium benzoate; 0.2-0.6%, preferably 0.4% of one or more water-soluble pH-modifying agents, such as citric acid; 0.2-0.8 of cosmetic agent, such as one or more of inulin and hyaluronic acid, preferably about 1% of inulin, about 0.25% of hyaluronic acid; and about 0.02-0.04%, preferably about 0.04 essential oils such as neroli maroc oil and 0.5-3%, preferably 1% of an emulsifier/dispersant such as caprylic ester and about 85% water.

In particular embodiments, the composition according to the invention is a solid form which, upon dissolution in water forms a face moisturizer product, said solid composition comprising 30-50% of a texturing agent comprising starch, such as sodium carboxymethyl potato starch; 3-8% water-soluble preservatives, such as potassium sorbate and sodium benzoate; 2.0-3.5% of one or more water-soluble pH-modifying agents, such as citric acid; 4.5-15% of a cosmetic agent, such as inulin and/or microalgae extract and/or plant-derived polysaccharide; and optionally between 30-50% of an aggregation controlling agent, such as maltodextrin; wherein the composition; 0.5-1.0% of essential oils such as geranium oil and 5-8% of an emulsifier such as caprylic ester. The application envisages different embodiments of the face moisturizer, wherein the amount of aggregation controlling agent is reduced as the amount of texturing agent is increased. Preferably, said solid form as detailed above can be dissolved in water to generate a moisturizing lotion. In particular embodiments, a solid form of about 7.5-10 g of said composition can be dissolved to a final volume of 50 ml to obtain a cosmetic such as a moisturizing lotion.

In particular embodiments, the composition according to the invention is a solid form which, upon dissolution in water, forms a face moisturizer product, said solid composition comprising 30-45% or 35-45% or 35-40%, preferably about 39% of a texturing agent comprising starch, such as sodium carboxymethyl potato starch; 30-45% or 30-40%, preferably about 35% of an aggregation controlling agent, such as maltodextrin; 3-6% water-soluble preservatives, preferably about 2% of two water-soluble preservatives such as potassium sorbate and sodium benzoate; 2.0-3.0%, preferably about 2.4% of one or more water-soluble pH-modifying agents, such as citric acid; 5.5-20% or 8-20%, preferably about 12% of one or more cosmetic agents, such as inulin plant-derived polysaccharide at between 5-6% each; wherein the composition also includes 0.5-1.0% of essential oils such as geranium oil (preferably 0.80%) and 5-8%, preferably about 6% of an emulsifier such as caprylic ester. Preferred embodiments comprise combinations of the preferred concentrations detailed herein. Preferably, said solid form as detailed above can be dissolved in water to generate a face moisturizer. In particular embodiments, a solid form of about 7-10 g of said composition can be dissolved to a final volume of 50 ml to obtain a cosmetic such as a face moisturizer.

In particular embodiments, the composition according to the invention is a solid form which, upon dissolution in water forms a face moisturizer product comprising 3-8%, preferably about 5% of an aggregation controlling agent, such as maltodextrin 4-9%, preferably about 6% of a texturing agent comprising starch, such as sodium carboxymethyl potato starch; 0.2-0.8%, preferably about 0.6 water-soluble preservatives, such as potassium sorbate and sodium benzoate; 0.2-0.5%, preferably about 0.35% of one or more water-soluble pH-modifying agents, such as citric acid; 05-2% of one or more cosmetic agents, such as inulin and/or microalgae extract and/or plant-derived polysaccharide such as about 0.7% of inulin and 0.8% of plant-derived polysaccharides; and 0.2-0.8%, preferably about 0.6% of essential oils such as geranium oil and 0.5-3%, preferably about 1% of an emulsifier such as caprylic ester and about 85% water. The application envisages different embodiments of the face moisturizer, wherein the amount of aggregation controlling agent is reduced as the amount of texturing agent is increased.

In particular embodiments, the composition according to the invention is a solid form which, upon dissolution in water forms a creamy sun screen product, said solid composition comprising 50-60% of a texturing agent comprising starch, such as sodium carboxymethyl potato starch; 4-8% water-soluble preservatives, such as potassium sorbate and sodium benzoate; 0.5-1.5% of one or more water-soluble pH-modifying agents, such as citric acid; 2-7.5% of a sun-protecting agent such as beta glucan and/or glucomannan or schizophyllan or hyaluronic acid/salt; 2-20% of an aggregation controlling agent, such as maltodextrin; 1-2% of essential oils such as tangerine and may chang oil and 7-10% of an emulsifier such as caprylic ester.

In particular embodiments, the composition according to the invention is a solid form which, upon dissolution in water, forms a make-up remover product, said solid composition comprising 37-47%, preferably about 43% of a texturing agent comprising starch, such as sodium carboxymethyl potato starch; 20-38%, preferably about 28% of an aggregation controlling agent, such as maltodextrin, 2-8%, preferably about 3.5% water-soluble preservatives, such as potassium sorbate and sodium benzoate; 4-8%, preferably about 6% of one or more water-soluble pH-modifying agents, such as citric acid; 10-20% of active agent, such as about 8% of disodium cocoyl glutamate and about 6% of inulin; 0.5-2%, such as about 1% of essential oils such as tangerine and litsee oil and 4-10%, 1 preferably about 6% of an emulsifier such as caprylic ester. Preferred embodiments comprise combinations of the preferred concentrations detailed herein. Preferably, said solid form as detailed above can be dissolved in water to generate a make-up remover. In particular embodiments, a solid form of about 9-10 g of said composition can be dissolved to a final volume of 50 ml to obtain a cosmetic such as a make-up remover. Preferably, the make-up remover product will comprise about 5% of an aggregation controlling agent such as maltodextrin, about 8% of a texturing agent such as sodium carboxymethyl potato starch, about 1% water-soluble pH-modifying agents such as citric acid, about 2.4% active ingredients such as inulin and disodium cocoyl glutamate, about 0.6% water soluble preservatives, about 0.15% essential oils and about 1% emulsifier and about 83% water.

In particular embodiments, the composition according to the invention is a solid form which, upon dissolution in water, forms a face cleanser product, said solid composition comprising 37-47%, preferably about 43% of a texturing agent comprising starch, such as sodium carboxymethyl potato starch; 15-35%, preferably about 22% of an aggregation controlling agent, such as maltodextrin, 2-8%, preferably about 3.6% water-soluble preservatives, such as potassium sorbate and sodium benzoate; 4-8%, preferably about 6% of one or more water-soluble pH-modifying agents, such as citric acid; 10-25% of active agent, such as about 8% of disodium cocoyl glutamate and about 6% of inulin; 0.5-2%, such as about 0.7% of essential oils such as eucalyptus and cardamom oil and 4-10%, preferably about 6% of an emulsifier such as caprylic ester. Preferred embodiments comprise combinations of the preferred concentrations detailed herein. Preferably, said solid form as detailed above can be dissolved in water to generate a face cleanser product. In particular embodiments, a solid form of about 8-10 g of said composition can be dissolved to a final volume of 50 ml to obtain a cosmetic such as a make-up remover. Preferably, the make-up remover product will comprise about 4% of an aggregation controlling agent such as maltodextrin, about 8% of a texturing agent such as sodium carboxymethyl potato starch, about 1% water-soluble pH-modifying agents such as citric acid, about 2% active ingredients such as disodium cocoyl glutamate and inulin, about 0.6% water soluble preservatives, about 0.1% essential oils and about 1% emulsifier and about 83% water.

In particular embodiments, the composition according to the invention is a solid form which, upon dissolution in water, forms a soft facial peeling product, said solid composition comprising 37-47%, preferably about 44% of a texturing agent comprising starch, such as sodium carboxymethyl potato starch; 15-35%, preferably about 23% of an aggregation controlling agent, such as maltodextrin, 2-8%, preferably about 3.6% water-soluble preservatives, such as potassium sorbate and sodium benzoate; 4-8%, preferably about 6% of one or more water-soluble pH-modifying agents, such as citric acid; 10-25% of active agent, such as about 8% of disodium cocoyl glutamate and about 7% of a scrubbing agent such as milled rice power; 0.5-2%, such as about 0.7% of essential oils such as eucalyptus and cardamom oil and 4-10%, preferably about 6% of an emulsifier such as caprylic ester. Preferred embodiments comprise combinations of the preferred concentrations detailed herein. Preferably, said solid form as detailed above can be dissolved in water to generate a face cleanser product. In particular embodiments, a solid form of about 8-10 g of said composition can be dissolved to a final volume of 50 ml to obtain a cosmetic such as a make-up remover. Preferably, the make-up remover product will comprise about 4% of an aggregation controlling agent such as maltodextrin, about 8% of a texturing agent such as sodium carboxymethyl potato starch, about 1% water-soluble pH-modifying agents such as citric acid, about 2.5% active ingredients such as disodium cocoyl glutamate and rice powder, about 0.6% water soluble preservatives, about 0.1% essential oils and about 1% emulsifier and about 83% water.

In particular embodiments, the composition according to the invention is a solid form which, upon dissolution in water, forms a pH balancing toner, said solid composition comprising 10-25%, preferably about 15% of a texturing agent comprising starch, such as corn starch; 50-75%, preferably about 65% of an aggregation controlling agent, such as maltodextrin, 2-8%, preferably about 4% water-soluble preservatives, such as potassium sorbate and sodium benzoate; 1-5%, preferably about 2% of one or more water-soluble pH-modifying agents, such as citric acid; 5-15% of active agent, such as about 7% of a hydrating prebiotic; 0.5-2%, such as about 1% of essential oils such as eucalyptus and cardamom oil and 4-10%, preferably about 6% of an emulsifier such as caprylic ester. Preferred embodiments comprise combinations of the preferred concentrations detailed herein. Preferably, said solid form as detailed above can be dissolved in water to generate a pH balancing toner product. In particular embodiments, a solid form of about 8-10 g of said composition can be dissolved to a final volume of 50 ml to obtain a cosmetic such as a pH balancing toner. Preferably, the pH balancing toner product will comprise about 10% of an aggregation controlling agent such as maltodextrin, about 3% of a texturing agent such as sodium carboxymethyl potato starch, about 0.3% water-soluble pH-modifying agents such as citric acid, about 1% active ingredients such as a hydrating prebiotic, about 0.6% water soluble preservatives, about 0.1% essential oils and about 1% emulsifier and about 85% water.

The solid form compositions of the present invention are envisaged for reconstitution in a given amount of liquid, so as to obtain a fluid cosmetic. The amount of the one or more cosmetic actives present in the composition is determined by the amount of cosmetic active envisaged in the reconstituted cosmetic product. Similarly, the amount of said water-soluble preservative, and optionally said water-soluble pH modifying agent and essential oil, in said solid form corresponds to the amount of said ingredients required for obtaining an active cosmetic product when reconstituted in a given amount of water. The solid forms as such are not suitable as cosmetics, given that they are solid and that the amount of cosmetic active agent is not suitable for direct use.

In particular embodiments, the solid form composition of the invention does not comprise an effervescent agent.

The compositions described herein are envisaged for external use only and as such are in most embodiments non-edible. The composition is in the form of a compact solid and does not contain water or other aqueous solutions.

In particular embodiments, said solid form has a given mass and is envisaged for reconstitution in a given amount of water. In particular embodiments, the solid form composition has a mass of between 5 and 25 grams and is envisaged for reconstitution in an amount of liquid between 5 and 1000 ml, preferably between 10 and 500 ml, more preferably between 10 and 250 ml or between 25 and 100 ml, or between 50 and 150 ml.

In particular embodiments, the weight ratio between the solid form and the final volume of the cosmetic product, addition of the given volume of water to the solid form, is between 4 and 12 times, preferably between 5 and 10, such as from 6.5 to 8.5. In particular embodiments the ratio is about 7 (corresponding to 8.23 g solid form for 50.25 g water). For instance, the tablet can be as small as 3-5 g for a final volume of cosmetic of 50 ml (ratio of about 10) but can also be between 8-10 g for a cosmetic product of 50 ml (ratio of about 5). In particular embodiments, the solid form is between 5-10 g for a final cosmetic product of about 50 ml, between 10-20 g for a final cosmetic product of about 100 ml, between 20-40 g for a final cosmetic product of about 200 ml . . . etc.

According to specific embodiments, the compositions in the solid form are dosed to be used as either 1 unit per 80-125 ml liquid, such as one unit per about 100 ml water. Alternatively, if a vial of 200 ml is used, 2 units may be required if the unit dose is 1 unit per 100 ml.

According to particular embodiments, the compositions described herein comprise at least one water-soluble cosmetic active in an amount between 0.11 and 10% in relation to the total weight of the composition, preferably between 3 and 6%, more preferably 1, 2, 3, 4, 5, 6, 7, 8, 9 or 10%. Upon dissolution of the composition in solid form in the specified volume of the liquid, a cosmetic product is obtained with suitable properties for use on the skin.

The solid form compositions of the invention are envisaged for reconstitution in water so as to obtain a cosmetic product. In particular embodiments said cosmetic is a skincare product, a haircare product or a bodycare product, whereby the ingredients of the solid form composition as described above are selected so as to ensure reconstitution in said skincare product, a haircare product or a bodycare product. In further particular embodiments, the viscous cosmetic product is selected from
(a) a make-up remover, such as a micellar make-up melt-away,
(b) a face cleanser, such as an ultra-gentle soap-free cleanser,
(c) a face toner, such as a pH-balancing toner,
(d) a face scrub, such as a microdermabrasion facial scrub,
(e) a face care product, such as an anti-oxidant moisturizing face care,
(f) an anti-pollution hydrating mist for face, hair and/or body mist,
(g) a deodorant,
(h) a hand and/or body soap,
(i) a solar skin protection product, such as a photo defense and bio protection product,
(j) an antimosquito product,
(k) a linnen spray,
(l) a face moisturizer, or face mask,
(m) a face serum, such as an intensive bio elixir with gorgeous plumping actives.

In preferred embodiments, the viscous cosmetic product is a face moisturizer or face mask or a face serum.

In particular embodiments, the viscous cosmetic product has a viscosity of a gel or a cream and preferred products are
a. a face and/or body cream, such as a hydrating cream,
b. a face and/or body scrub, or
c. a solar skin protection cream.

The nature of the solid form of the solid form composition of the invention is not critical. Envisaged forms of the solid-form composition include but are not limited to a tablet, a cylinder, a sphere, a cube, a losange etc. In particular embodiments, the shape is that of a cylinder.

In particular embodiments the solid form is a cylinder with a diameter of between 1 and 4 cm, preferably between 2 and 3 cm and a height of between 4 and 10 cm, particularly between 4-6 cm. In particular embodiments, the diameter is at least 1.5 cm.

In particular embodiments the solid form is a sphere with a diameter of between 1 and 7 cm, preferably between 2 and 3 cm. In particular embodiments, the diameter is at least 1.5 cm.

In particular embodiments, the solid form is in the shape of a tablet, with a height of at least 2 mm and a diameter of at least 5 mm. In a particular embodiment, the solid form is in the form of a cylinder with dimensions of about 1.0 to 3.0 cm in diameter, a height between 1.0 and 4.0 in height and a volume ranging from between 0.9 to 28.3 cm³. In a most preferred embodiment a cylindrical tablet is envisaged having a diameter of about 2.0 cm, a height between 2.25 and 3.06 cm and a volume ranging from between 7.07 to 9.62 cm³.

The solid-form composition of the present invention further has a number of characteristics which are of interest in the context of the present invention. Particularly, the properties of the solid form composition are such that they promote dispersion or emulsion of the composition in the fluid, when contacted with said fluid.

Further features of the solid-form compositions of the present invention include compactness, not irritating, not greasy and firm at room temperature (not melting) (easily handled) homogeneity, solidity, solubility in fluid, such as water, etc.

A further aspect of the present invention relates to methods for producing a cosmetic product, using the solid form compositions of the present invention.

The invention thus also relates to a method for producing a cosmetic product, said method comprising dispersing a composition in solid form as described herein in a given amount of fluid. More particularly, the composition comprises a cosmetic active in a concentration corresponding to the concentration required for said cosmetic active in the final cosmetic product. Accordingly, when the solid form composition is dispersed in a given volume, said cosmetic is reconstituted. Dispersing the solid form composition preferably occurs in a vial, more particularly a vial which is suitable for holding and dispensing the cosmetic product after reconstitution. In particular embodiments, the predetermined amount of fluid, such as water, is introduced into the vial first, after which the solid form composition is added to the vial. Alternatively, the solid form composition is introduced into the vial first, after which the fluid is added. In particular embodiments, the method may comprise shaking the vial so as to promote dispersing or emulsion of the solid form composition. In particular embodiments, the method may comprise stirring with a stirring tool in the vial to further dissolution of the solid form composition. In particular embodiments, the method may comprise grinding the solid-form composition with a grinding tool. In particular embodiments, the method comprises one or more of the steps described above.

In particular embodiments, the fluid is a water-based composition such as tap water, bottled water, distilled water, de-ionized water, sparkling water.

In particular embodiments, the fluid is a buffered solution.

The compositions are characterized in that the resulting products can be reconstituted by simply dissolving the composition in solid form in a fluid requiring only simple manual mixing.

As detailed above, the solid-form compositions of the present invention are envisaged for reconstitution in a given volume of liquid so as to obtain a cosmetic product, preferably a viscous cosmetic product such as a gel or a cream. In order to ensure reconstitution in the right amount of fluid, appropriate vials can be provided which (a) allow easy reconstitution of the solid form compositions of the invention and optionally (b) allow easy proportioning of the reconstituted cosmetic.

Accordingly, a further aspect of the invention provides a kit, comprising the compositions of the present invention and a vial, more particularly a vial suitable for reconstitution of the cosmetic, i.e. which ensures that the composition can be reconstituted in the appropriate volume of fluid. In particular embodiments, the vial is an empty vial which can be filled with the appropriate amount of the suitable liquid. More than one solid-form compositions can be reconstituted consecutively using the same vial, allowing a reduction in packaging. Hence, in a particular embodiment, the kit of the present invention comprises two or more of the compositions in solid form according to the invention and at least one empty vial or container suitable for holding a fluid cosmetic.

In particular embodiments, the vial contains a demarcation indicating the level of the fluid, for the given volume of fluid when provided in the vial.

Preferably, at least one vial is provided together in the kit together with one or more of the compositions in solid form. More preferably 6, 12 or 24 compositions in solid form are provided in one kit. In particular embodiments, the vial is an empty vial.

The vial provided in the kit described herein is re-fillable. The vial may be proportioned to contain a volume between 50 and 1000 ml, preferably between 100 and 500 ml, or between 150 and 250 ml. The vial may contain any volume as necessary for reconstituting and holding a fluid cosmetic. The vial may be in glass, in ceramic, in stainless steel, made of plant based-plastic, or may be a mix of 2 or 3 materials.

In particular embodiments, the vial allows for easy dispensing of the fluid cosmetic. The shape of the vial is not critical. However, in particular embodiments, the opening of the vial is such that it allows for easy passage of the solid form composition into the vial. In further particular embodiments, the vial allows portioned dispensing of the cosmetic composition. Typically, this implies the provision of a dispenser cap or vial head, such as pump-dispenser. Thus in particular embodiments, the kit of the present invention further comprises a vial head, more particularly a pump or a roll-on head. The kits described herein may thus comprise at least one vial head, which is adapted to be used in combination with the cosmetic product.

In particular embodiments, the vial comprises a demarcation as to the appropriate level of fluid that is to be used for reconstitution of the fluid cosmetic. In further embodiments, the vial comprises multiple demarcations corresponding to the reconstitution of one or more volumes of the fluid cosmetic to be reconstituted. Typically the demarcation is a visual element on the side of the vial, such as a horizontal line or cross.

In particular embodiments, the kit comprises more than one vial head, such that an appropriate vial head can be selected.

Though typically the compositions of the invention can be dissolved by manual shaking, in particular embodiments, the kit may comprise a device for grinding the solid-form composition of the invention and/or for stirring in the vial, so as to promote dispersion or emulsion of the composition in the vial. In particular embodiments, the device is a combined tool for grinding and stirring. In particular embodiments, the device has a length which allows it to be introduced towards the bottom of the vial, i.e. its length is longer than that of the vial present in the kit.

In particular embodiments, the kit comprises more than one empty vial and more than one composition in solid form as described herein. The more than one composition in solid form can be the same or can be different. For instance, two different compositions in solid form, each for a distinct cosmetic application, are present in the kit. For instance the kit may comprise at least two, preferably at least three, at least four or at least five distinct compositions in solid form selected from compositions which can be reconstituted to a face, hand and/or body cream or gel, make-up remover, a face cleanser, a face toner, a face scrub, a face care product, an anti-pollution hydrating mist for face, hair and/or body, a deodorant, a hand and body soap, a solar skin protection product, an anti-mosquito product, and a face serum.

In particular embodiments, the kit comprises an additional container comprising a liquid suitable for reconstitution of the solid-form composition of the invention into a cosmetic as described herein. Preferably, the container is of a size such that it contains sufficient fluid for the reconstitution of more than one solid composition of the present invention.

In particular embodiments of the kits according to the present invention, a leaflet is provided, which describes how the solid-form composition of the invention can be reconstituted to a cosmetic product using the vial and a fluid as described herein, using the method for the production/reconstitution of a cosmetic as described herein. Preferably, the leaflet describes one or more of the solid-form composition, the vial, the vial head and the container present in the kit.

A further aspect of the invention relates to methods of producing the solid form composition of the present invention described herein. More particularly, the invention provides methods for producing solid form compositions suitable for reconstitution to a cosmetic.

In particular embodiments said method for producing the solid form cosmetic product comprises grinding, blending, pressing and unmolding steps. In further particular embodiments, the methods comprise the steps of:
(a) grinding the ingredients of the solid form composition as described herein having particle size larger than 0.2 mm,
(b) blending the ingredients to a homogenous mass,
(c) providing the homogenous mass in a mold,
(d) pressing the mass in the mold, and
(e) unmolding the solid form.

Depending on the nature of the composition, at least some of the ingredients are particulate (provided as powder or granules), while others are provided as liquid/oil. Preferably, most of the ingredients are provided in solid form, i.e. as particulate. According to particular embodiments of the invention, the grinding of the particular ingredients is done using a grinder. The grinder may be any grinder known in the art, such as for example a MultiDrive basic grinder from IKA, or a larger grinder for industrial use.

In particular embodiments, the methods of the invention further comprise mixing of the oily ingredients separately, whereafter the blended oily mixture is blended with the ground ingredients to a homogeneous paste.

The molds used in the context of the present invention ensure the shape of the solid-form compositions of the present invention. The mold die can be any mold known in the art, suitable for molding a homogeneous mass made up of the ingredients described herein.

The step of pressing the mass in the mold is typically ensured using a press. In particular embodiments, the mass is pressed in the mold using higher than atmospheric pressure, preferably a pressure of about 100-1000 kg.

In particular embodiments, the method step (d) described above comprises pressing the mass with a stamp press so as to obtain a logo, a brand or a text on the solid form. Alternatively the logo, brand or text can be printed on the solid form in a separate step after step (d) or (e) of the method described above.

In particular embodiments, the method may comprise a quality control step which involves checking one or more of the shape, density, homogeneity, solidity, solubilization time of the solid form composition. In particular embodiment, the method comprises determining that the composition is capable of being completely dissolved in water at room temperature in a time period between 0-30 min.

In particular embodiments, the production of the solid form does not require special techniques, special molds, intense forces, or temperature control. Indeed the preparation of the solid form compositions requires only pressure (that can be mechanical with a lever or an actuator) and simple molds (i.e. steel undercutting is enough, the shapes typically do not require complicated design and the number of pieces of the mold are reduced to three: punch, matrix, bottom. This is in contrast to complicated molds, which require the opening of several pieces to free the tablet, or which require steel guides to bring the punch to the right place. This is an important cost-advantage and logistics advantage of the production methods of the present invention.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and scope of the appended claims.

The herein disclosed aspects and embodiments of the invention are further supported by the following non-limiting examples.

### EXAMPLES

### Example 1: Formulation of a Solid-Form Composition which can be Reconstituted into a Make Up Remover Gel Product with a Given Amount of Water

For an active cosmetic which is a cleaning product which hydrates the skin and protects useful commensal bacteria, the following ingredients were combined (percentages are rounded off):
texturing agent: sodium carboxymethyl potato starch 56%
aggregation controlling agent: maltodextrin
active with hydration effect and protective effect for useful commensal bacteria: inulin: 8%+Sodium cocoyl glutamate 10%
pH modifying agent: citric acid: 1%
essential oils: 1.3% (Tangerine and May Chang)
emulsifier: caprylic ester or polyglycery-3-caprylate: 10%
preservatives: potassium sorbate, sodium benzoate: 5%

The solid ingredients are ground in a grinder; the liquid or pasty ingredients (essential oils, sodium cocoyl glutamate) are mixed together. The ground ingredients are blended with the remainder of the ingredients to a homogenous mass. The homogenous mass is then provided in a tablet mold, and the mass is then pressed in the mold.

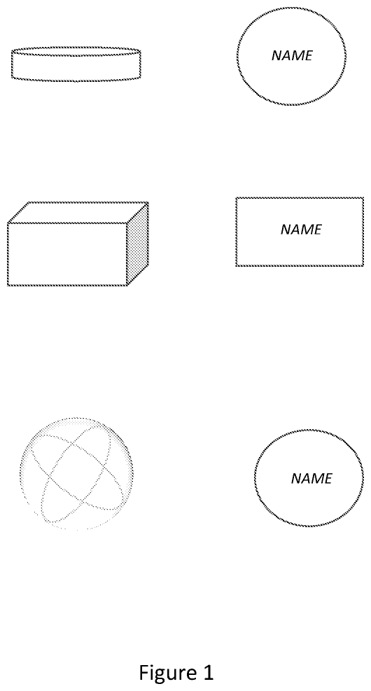

Finally, the solid form is unmolded so as to obtain the solid form of 1 g. Examples of solid forms are provided in FIG. 1.

The resulting product when dissolved in a corresponding amount of water (7.23 ml) generates 8.23 ml of a fluid gel, slightly foaming, pH 5+/−0.1

### Example 2: Formulation of a Solid-Form Composition which can be Reconstituted into a Cleanser Gel Product with a Given Amount of Water

The following ingredients were combined:
texturing agent: sodium carboxymethyl potato starch 56%
active with hydration effect and protective effect for useful commensal bacteria: inulin: 8%+sodium cocoyl glutamate 15%
pH modifying agent: citric acid: 1%
essential oils: 1.4% (Mint and Bergamot)
emulsifier: Caprylic methyl ester: 10%
preservatives: potassium sorbate, sodium benzoate: 5%

The solid ingredients are ground in a grinder; the liquid or pasty ingredients (essential oils, sodium cocoyl glutamate) are mixed together. The ground ingredients are blended with the remainder of the ingredients to a homogenous mass. The homogenous mass is then provided in a tablet mold, and the mass is then pressed in the mold.

Finally, the solid form is unmolded so as to obtain the solid form of 6.95 g. Examples of solid forms are provided in FIG. 1. Reconstitution could be obtained with 50.25 ml of water to obtain 54.98 ml of a fluid gel, which foams on the hands.

### Example 3: Further Cosmetic Formulations

(a) for a First Cosmetic the Following Ingredients were Combined
texturing agent: sodium carboxymethyl potato starch 48%
aggregation controlling agent: maltodextrin 27%
active with skin-treatment properties: inulin 5%, algae extract 0.7%, nicotinamide 4.8%, other actives 1.5%
pH modifying agent: citric acid: 0.8%
essential oils: 0.8% (geranium)
emulsifier: Caprylic methyl ester: 6%
preservatives: potassium sorbate, sodium benzoate: 4%

The solid ingredients are ground in a grinder; the liquid or pasty ingredients (essential oils, emulsifier) are mixed together. The ground ingredients are blended with the remainder of the ingredients to a homogenous mass. The homogenous mass is then provided in a tablet mold, and the mass is then pressed in the mold.

Finally, the solid form is unmolded so as to obtain the solid form of 8.34 g. Examples of solid forms are provided in FIG. 1. Reconstitution could be obtained with 50.25 ml of water to obtain 58.6 ml of a the cosmetic product, i.e; a dilution of about 7%.

(b) for a Second Cosmetic the Following Ingredients were Combined
texturing agent: sodium carboxymethyl potato starch 54%
aggregation controlling agent: maltodextrin 32%
active with skin-treatment properties: algae extract 0.7%, other actives 0.7%
pH modifying agent: citric acid: 0.8%
essential oils: 0.8% (geranium)
emulsifier: Caprylic methyl ester: 7%
preservatives: potassium sorbate, sodium benzoate: 4%

The solid ingredients are ground in a grinder; the liquid or pasty ingredients (essential oils) are mixed together. The ground ingredients are blended with the remainder of the ingredients to a homogenous mass. The homogenous mass is then provided in a tablet mold, and the mass is then pressed in the mold.

Finally, the solid form is unmolded so as to obtain the solid form of 8.34 g. Examples of solid forms are provided in FIG. 1. Reconstitution could be obtained with 50.25 ml of water to obtain 58.6 ml of a the cosmetic product, i.e. a dilution of about 7%.

### Example 4: Formulation of a Solid-Form Composition which can be Reconstituted into a Scrub Product with a Given Amount of Water

The following ingredients were combined:
texturing agent: sodium carboxymethyl potato starch 56%-active for scrubbing and optionally with soothing agent: grains 10-21%+optionally dipotassium glycyrrhizinate 8%
pH modifying agent: citric acid: 1%
essential oils: 1. % eucalyptus and cardamom)
emulsifier: caprylic methyl ester: 10%
preservatives: potassium sorbate, sodium benzoate: 5%

The solid ingredients (except for the grains) are ground in a grinder; the liquid or pasty ingredients (essential oils, dipotassium glycyrrhizinate) are mixed together. The ground ingredients are blended with the remainder of the ingredients to a homogenous mass, to which the grains are added. The homogenous mass is then provided in a tablet mold, and the mass is then pressed in the mold.

Finally, the solid form is unmolded so as to obtain the solid form of 6.95 g. Examples of solid forms are provided in FIG. 1. Reconstitution could be obtained with 50.25 ml of water to obtain 54.98 ml of a face and/or body scrub.

### Example 5: Formulation of a Solid-Form Composition which can be Reconstituted into a Face Moisturizer Product with a Given Amount of Water

The following ingredients were combined:
texturing agent: sodium carboxymethyl potato starch 39%
aggregation controlling agent: maltodextrin 35%
cosmetic active: Inulin 5% and optionally microalgae extract 1% and optionally plant-derived polysaccharide 6%
pH modifying agent: citric acid: 2%
essential oils: 1. % geranium oil
emulsifier: caprylic ester: 7%
preservatives: potassium sorbate, sodium benzoate: 4%

Finally, the solid form is unmolded so as to obtain the solid form of 7.93 g. Examples of solid forms are provided in FIG. 1. Reconstitution could be obtained with 50 ml of water to obtain 44.22 ml of a face moisturizer.

### Example 6: Formulation of a Solid-Form Composition which can be Reconstituted into a Serum Product with a Given Amount of Water

The following ingredients were combined:
texturing agent: sodium carboxymethyl potato starch 41%
aggregation controlling agent: maltodextrin 37%
cosmetic active: Inulin 7% and optionally Hyaluronic acid 1.5% and Sodium Retinoyl Hyaluronate 0.5%
pH modifying agent: citric acid: 2%
essential oils: neroli maroc oil 0.5%
emulsifier: caprylic ester: 6.5%
preservatives: potassium sorbate, sodium benzoate: 4%

Finally, the solid form is unmolded so as to obtain the solid form of 7.74 g. Examples of solid forms are provided in FIG. 1. Reconstitution could be obtained with 50 ml of water to obtain 44.75 ml of a serum.

### Example 6: Making of a Viscous Cosmetic Using the Composition in Solid Form According to the Invention

The compositions of the invention produced as described in example 1 or 2 are used for the production of a more or less viscous cosmetic. More particularly, a solid tablet of 1 gram is provided in a suitable vial comprising 7.23 ml of water. More particularly, an empty 100 ml vial is filled with 7.23 ml deionized water, after which the solid form composition of example 1 is introduced therein. The vial is closed with a cap or vial head and shaken to promote dissolution of the solid form composition into the cosmetic product. Alternatively, compositions of 6.95 g solid form of example 3 are reconstituted with 50.25 ml of water to obtain a cosmetic product with the desired viscosity and relative amount of active product.

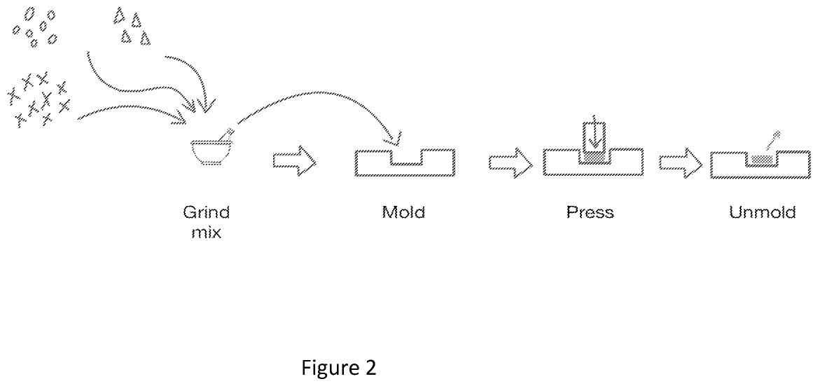

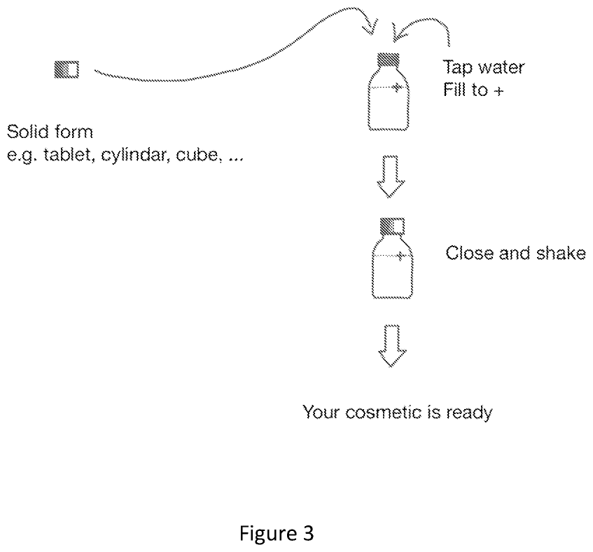

An exemplary method is described in FIG. 2.

### Example 7: Method of Making a Composition in Solid Form which is Stable but can be Dissolved in a Given Amount of Water into a Cosmetic Product with a Given Viscosity

Compositions are provided which comprise varying amounts of a texturing agent comprising one or more cold-dispersible modified starches, a one or more aggregation controlling agents and a texturing agent. This combination of agents allows for the production of solid form cosmetics which, when one or more cosmetic actives are added thereto in the right concentration, can be dissolved into a cosmetic product with a specified amount of water at a dilution rate of 8.23.

Viscosity was measured by an IKA ROTAVISC SBS, using spindles sp2+sp3+sp4 at temperatures of 20±2 °C.

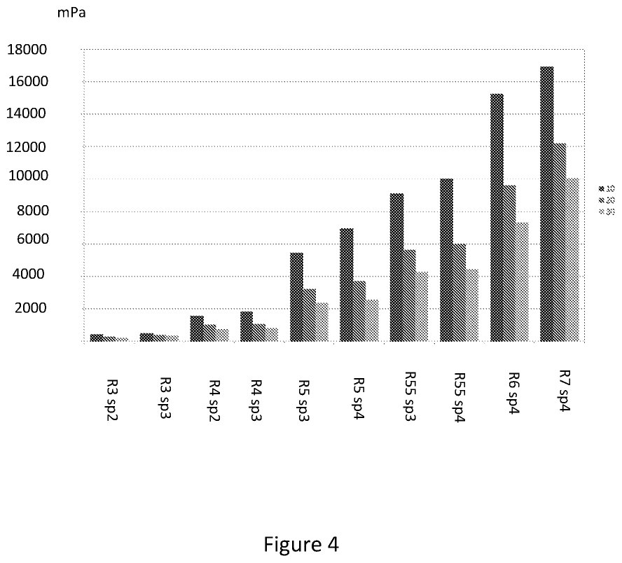

More particularly, experiments were carried out with acetylated distarch phosphate (R). Increasing concentrations of the cold-soluble modified starch generated solid forms which could be dissolved at a weight ratio of 8.23 in water to cosmetics with increasing viscosity. This is illustrated in FIG. 4. In FIG. 4, varying concentrations of R are used (the weight % of R corresponding to the diluted form in water; R3=3%; R4=4%, R5=5%, R5.5=5.5%, R6=6%, R7=7% corresponding to concentrations in the tablet between 24.7% to 57.6%). The viscosity is tested with increasingly small spindles (sp2-sp4), at different rpms (10, 20, 30). The viscosity (in mPas) evolves from a quite fluid composition, at a concentration of R3=3%, to a quite viscous one, at R7=7%, of acetylated distarch phosphate in water.

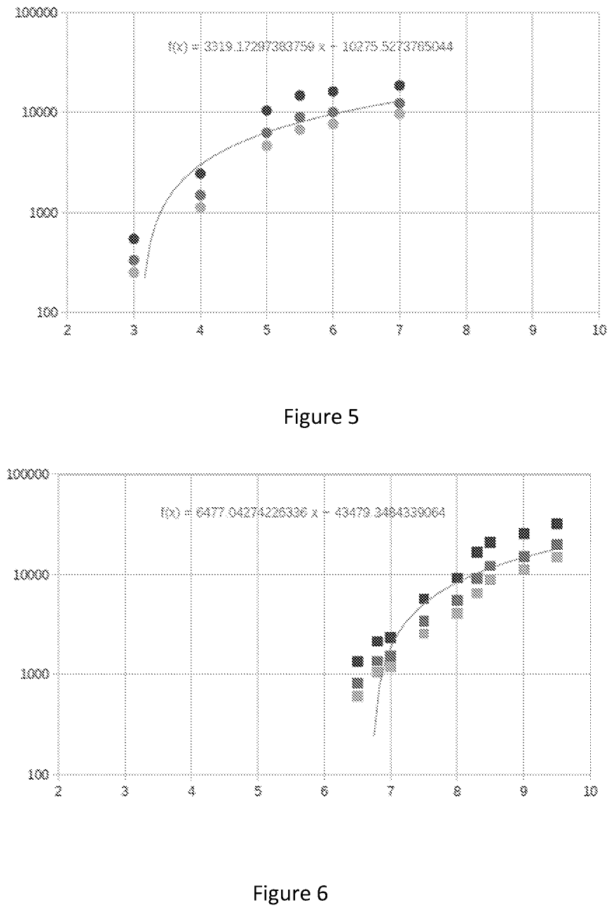

The viscosity obtained with acetylated distarch phosphate (R) was compared to that obtained with sodium carboxymethyl starch(S). FIGS. 5 and 6 demonstrate that a similar range in viscosity can be obtained when using increasing concentrations of acetylated distarch phosphate (R) (FIG. 5) and sodium carboxymethyl starch(S) (FIG. 6). X-axis represents % weight of starch at 10-20-30 rpm (represented by increasingly dark shades of data points), Y-axis represents mPas log scale.

The inventors have found that cold-dispersible modified starches are particularly suitable as an ingredient of compositions which are to be provided in solid form, but that the amount used can be varied for adjusting the texture of the final cosmetic product. For instance, for obtaining fluid gels, a concentration of between 50-60% of the cold-dispersible modified starch is used. For obtaining a mask around 70% of the cold-dispersible modified starch is used.

The following compositions were obtained, with differing viscosity (values in weight %):

TABLE 1
exemplary compositions

| | | Aggregation | | | PH-modifying | Emulsifier/ | |
|---|---|---|---|---|---|---|---|
| | Texturing agent | controlling agent | Active | Preservative | agent | dispersant | Oil |
| Make-up | Sodium | Maltodextrin 10.46 | Inulin 8.23 | KSorb 2.47 | Citric acid 1.02 | Caprylic ester | Tangerine 1.23 |
| removing gel | carboxymethyl | | Sodium cocoyl | NaBenz 2.47 | | 8.38 | Litsee 0.08 |
| | potato starch | | glutamate 10 | | | | |
| | 55.65 | | | | | | |
| Make up | Sodium | Maltodextrin 28.2 | Inulin 5.67 | KSorb 1.7 | Citric acid 5.85 | Caprylic ester | Tangerine 0.85 |
| removing gel 2 | carboxymethyl | | Sodium cocoyl | NaBenz 1.7 | | 5.67 | Litsee 0.06 |
| | potato starch | | glutamate 7.69 | | | | |
| | 42.78 | | | | | | |
| Cleanser gel | Sodium | Maltodextrin 5.3 | Inulin 8.23 | KSorb 2.47 | Citric acid 1.02 | Caprylic ester | Menthe 0.82 |
| | carboxymethyl | | Sodium cocoyl | NaBenz 2.47 | | 8.38 | Bergamot 0.61 |
| | potato starch | | glutamate 15 | | | | |
| | 55.64 | | | | | | |
| Cleanser 2 | Sodium | Maltodextrin 22.05 | Inulin 5.79 | KSorb 1.74 | Citric acid 6.72 | Caprylic ester | Menthe 0.58 |
| | carboxymethyl | | Sodium cocoyl | NaBenz 1.74 | | 5.79 | Bergamot 0.46 |
| | potato starch | | glutamate 11.71 | | | | |
| | 43.43 | | | | | | |

| | | Aggregation | | | PH-modifying | | |
|---|---|---|---|---|---|---|---|
| | Texturing agent | controlling agent | Active | Preservative | agent | Emulsifier | Oil |
| Scrub1 | Sodium | — | Dipotassium | KSorb 2.47 | Citric acid 1.02 | Caprylic ester | Eucalyptus 0.8 |
| | carboxymethyl | | glycyrrhizinate | NaBenz 2.47 | | 8.38 | Cardamom 0.16 |
| | potato starch | | 8.2 | | | | |
| | 55.64 | | rice exfoliating | | | | |
| | | | grains 20.79 | | | | |
| Scrub2 | Sodium | Maltodextrin 8.2 | rice exfoliating | KSorb 2.47 | Citric acid 1.02 | Caprylic ester | Eucalyptus 0.8 |
| | carboxymethyl | | grains | NaBenz 2.47 | | 8.38 | Cardamom 0.16 |
| | potato starch | | 20 | | | | |
| | 55.64 | | | | | | |
| Scrub3 | Sodium | Maltodextrin 19.025 | rice exfoliating | KSorb 2.47 | Citric acid 1.02 | Caprylic ester | Eucalyptus 0.8 |
| | carboxymethyl | | grains 10 | NaBenz 2.47 | | 8.38 | Cardamom 0.16 |
| | potato starch | | | | | | |
| | 55.64 | | | | | | |
| Scrub4 (best | Sodium | Maltodextrin 9.025 | rice exfoliating | KSorb 2.47 | Citric acid 1.02 | Caprylic ester | Eucalyptus 0.8 |
| version) | carboxymethyl | | grains 10 | NaBenz 2.47 | | 8.38 | Cardamom 0.16 |
| | potato starch | | Sodium cocoyl | | | | |
| | 55.64 | | glutamate 10 | | | | |
| Face peeling | Sodium | Maltodextrin 23.03 | rice exfoliating | KSorb 1.8 | Citric acid 6.27 | Caprylic ester | Eucalyptus 0.60 |
| | carboxymethyl | | grains 7.33 | NaBenz 1.8 | | 6.00 | Cardamom 0.12 |
| | potato starch | | Sodium cocoyl | | | | |
| | 44.96 | | glutamate 8.08 | | | | |
| Mask1 | Sodium | Maltodextrin 2.27 | agave tequilana | KSorb 2.47 | Citric acid 1.02 | Caprylic ester | Tangerine 1.23 |
| | carboxymethyl | | extract 11.118 | NaBenz 2.47 | | 8.38 | Litsee 0.08 |
| | potato starch | | | | | | |
| | 70.94 | | | | | | |
| Mask 2 | Sodium | Maltodextrin 17.57 | agave tequilana | KSorb 2.47 | Citric acid 1.02 | Caprylic ester | Tangerine 1.23 |
| | carboxymethyl | | extract 11.118 | NaBenz 2.47 | | 8.38 | Litsee 0.08 |
| | potato starch | | | | | | |
| | 55.64 | | | | | | |
| Mask 3 (best | Sodium | Maltodextrin 7.57 | agave tequilana | KSorb 2.47 | Citric acid 1.02 | Caprylic ester | Tangerine 1.23 |
| version) | carboxymethyl | | extract 11.118 | NaBenz 2.47 | | 8.38 | Litsee 0.08 |
| | potato starch | | Sodium cocoyl | | | | |
| | 55.64 | | glutamate 10 | | | | |
| | | Aggregation | | | | | |
| | | controlling | | | | | |
| | | agent/desintegrating | | | PH-modifying | | |
| | Texturing agent | agent | Active | Preservative | agent | Emulsifier | Oil |
| Face wash | — | Maltodextrin 51 | Aloe vera 0.4 | KSorb 2.47 | Citric acid 1.02 | Caprylic ester | Eucalyptus 0.8 |
| | | Cornstarch 20 | Inulin 12 | NaBenz 2.47 | | 8.38 | Cardamom 0.2 |

| | | Aggregation | | | PH-modifying | | |
|---|---|---|---|---|---|---|---|
| | Texturing agent | controlling agent | Active | Preservative | agent | Emulsifier | Oil |
| body wash | Sodium | Maltodextrin 18.6 | Ethyl ascorbic | KSorb 2.47 | Citric acid 1.02 | Caprylic ester | Tangerine 1.23 |
| | carboxymethyl | | acid 0.8% | NaBenz 2.47 | | 8.38 | Litsee 0.08 |
| | potato starch | | Aloe vera 200 | | | | |
| | 55.64 | | 0.4% | | | | |
| | | | Sodium cocoyl | | | | |
| | | | glutamate 7.5% | | | | |
| Soap for the | Sodium | Maltodextrin 15 | Sodium cocoyl | KSorb 2.47 | Citric acid 1.02 | Caprylic ester | Tangerine 1.23 |
| body and face | carboxymethyl | | glutamate 12% | NaBenz 2.47 | | 8.38 | Litsee 0.08 |
| | potato starch | | | | | | |
| | 55.64 | | | | | | |
| Hair care | Sodium | Maltodextrin 15 | Sodium cocoyl | KSorb 2.47 | Citric acid 1.02 | Caprylic ester | Neroli Tunesia |
| product | carboxymethyl | | glutamate 12% | NaBenz 2.47 | | 8.38 | 0.3 |
| | potato starch | | | | | | |
| | 55.64 | | | | | | |
| deodorant | Sodium | Maltodextrin 10 | Prebiulin fos 8.3 | KSorb 2.47 | Citric acid 1.02 | Caprylic ester | Geranium 2 |
| | carboxymethyl | | Sodium cocoyl | NaBenz 2.47 | | 8.38 | |
| | potato starch | | glutamate 10% | | | | |
| | 55.64 | | | | | | |
| Sunscreen | Sodium | Maltodextrin 3.5 | Inulin 11 | KSorb 2.47 | Citric acid 1.02 | Caprylic ester | Neroli Egypt 1.0 |
| | carboxymethyl | | glucomannan | NaBenz 2.47 | | 8.38 | Tangerine 1.4 |
| | potato starch | | 2.5 | Sodium cocoyl | | | |
| | 55.64 | | | glutamate 10% | | | |
| Face | Sodium | Maltodextrin 35.12 | Inulin 4.84 | KSorb 1.99 | Citric acid 2.35 | Caprylic ester | Geranium 0.80 |
| Moisturizer | carboxymethyl | | microalgae | NaBenz 1.99 | | 6.49 | |
| | potato starch | | extract 0.66 | | | | |
| | 38.73 | | plant-derived | | | | |
| | | | polysaccharide | | | | |
| | | | 5.57 | | | | |
| Serum | Sodium | Maltodextrin 36.49 | Inulin 6.79 | KSorb 2.04 | Citric acid 2.77 | Caprylic ester | Neroli maroc |
| | carboxymethyl | | Hyaluronic acid | NaBenz 2.04 | | 6.63 | 0.27 |
| | potato starch | | 1.36 | | | | |
| | 41.13 | | Sodium Retinoyl | | | | |
| | | | Hyaluronate | | | | |
| | | | 0.34 | | | | |
| pH balancing | Zea Mays (Corn) | Maltodextrin 64.38 | Inulin 6.49 | KSorb 1.95 | Citric acid 1.89 | Caprylic ester | Eucalyptus 0.66 |
| toner | Starch 15.77 | | Aloe vera | NaBenz 1.95 | | 6.49 | Cardamom 0.13 |
| | | | powder 0.32 | | | | |

1. A solid form comprising, in weight percentage a composition comprising:
30-70% of a texturing agent comprising one or more cold-dispersible modified starches;
1-10% water-soluble preservatives;
0.1-10% of one or more water-soluble pH-modifying agents; and
0.1-30% of one or more water-soluble and dispersible cosmetic actives; and, optionally
0.1-3% essential oils;
5-10% of emulsifier;
0-50% of one or more aggregation controlling agents;
characterized in that said solid form is suitable for dissolving in a given volume of water in a vial so as to obtain a viscous cosmetic product.

2. The solid form according to claim 1, wherein said texturing agent is a modified starch such as sodium carboxymethyl starch, acetylated distarch adipate (63798-35-6) or acetylated distarch phosphate (68130-14-3).

3. The solid form according to claim 1, comprising, in weight percentage
30-50% of a texturing agent comprising one or more cold-dispersible modified starches;
3-8% water-soluble preservatives;
2.5-3.5% of one or more water-soluble pH-modifying agents;
4.5-15% of one or more water-soluble and dispersible cosmetic actives;
0.1-1.0% essential oils;
5-8% of emulsifier; and
30-50% of one or more aggregation controlling agents, preferably maltodextrin;
characterized in that said composition is in solid form for dissolving in a given volume of water so as to obtain a viscous cosmetic product.

4. The solid form according to claim 1, wherein the amount of said water-soluble preservative, water-soluble pH modifying agent, and said water-soluble and dispersible cosmetic actives in said solid form corresponds to the amount of said ingredients required for obtaining an active creamy and/orviscous cosmetic product when reconstituted in said given volume of water.

5. The solid form according to claim 4, wherein said solid form has a mass of between 2 and 50 grams, and said given amount of water is between 10 and 250 ml, preferably wherein said solid form has a mass of preferably between 5-10 grams and said given volume of water is between 35 and 55 ml.

6. The solid form according to claim 1, wherein said one or more cosmetic actives are selected from low, medium or high molecular weight polysacharides, beta glucan, sodium (retinoyl) hyaluronate, bisabolol, niacinamide, biosaccharide gum-1, haematococcus pluvialis extract, panthenol, aloe vera extract, agave extract, allantoin, inulin, zinc salts, and copper salts, prebiotics and scrubbing agents.

7. The solid form according to claim 1, wherein said cosmetic active comprises a skin cleaning agent such as sodium cocoyl glutamate, wherein when a concentration of more than 10% of sodium cocoyl glutamate is present, the aggregation controlling agent is present in less than 25%.

8. The solid form according to claim 1, wherein
said aggregation controlling agent is a maltodextrine,
said texturing agent is sodium carboxymethyl starch,
said emulsifier is caprylic acid,
the water-soluble preservative is selected from one or more of potassium sorbate, sodium benzoate, benzoic acid, levulinic acid, dehydroacetic acid, sorbic acid, and p-anisic acid; and
the pH modifying agent is citric acid.

9. The solid form according to claim 1, wherein said one or more essential oils are selected from essential oils of cardamom, eucalyptus, tangerine, neroli, mint, bergamot, litchee, and geranium.

10. The solid form according to claim 1, wherein said composition further comprises a dispersing agent such as a sugar.

11. The solid form according to claim 1, wherein said solid form is a tablet, sphere, a cylinder or a block.

12. The solid form according to claim 1, wherein the dissolution of the solid form in water results into one of the following viscous cosmetic products:
a. a face and/or body cream, serum or lotion, such as a hydrating cream, serum or lotion
b. a face and/or body scrub, or
c. a solar skin protection cream.

13. The solid form according to claim 1, wherein the addition of the given volume of water to the solid form results in a cosmetic product with a weight that is between 5 and 10 times that of the solid form after addition of the given volume of water to the solid form.

14. A kit comprising two or more of the solid form of claim 1 and at least one empty vial suitable for holding and optionally dispensing a viscous cosmetic product resulting from the addition of said given volume of water to said solid form, optionally further comprising a vial head suitable for dispensing the viscous cosmetic product.

15. A consumer-friendly method for producing a creamy cosmetic or cleansing product, said method comprising dissolving a solid form according to claim 1, comprising said cosmetic active in a concentration corresponding to the concentration required for said cosmetic and/or cleansing product in a given volume, in a volume of water corresponding to said given volume.

16. A method for producing the solid form cosmetic product according to claim 1 comprising
(a) Grinding the ingredients having particle size larger than 0.5 mm; optionally also grinding ingredients having a particle size larger than 0.2 mm;
(b) Blending the ingredients to a homogenous mass;
(c) Providing the homogenous mass in a mold;
(d) Pressing the mass in the mold under higher than atmospheric pressure; and
(e) Unmolding the solid form:
Preferably wherein step (d) comprises pressing the mass with a stamp press so as to obtain a logo or a text on the solid form.
